SEQUENCE LISTING (15 sequences)

SEQ ID NO: 1 - DNA, 1695 bp, Artificial Sequence
  nucleotide sequence of Tpol
atggctagcgccgaaggttttgaactgcattatattccggaagttggtccgggtatgggtgaactgctggatctgctgatgcgtcagccggttctgggtgttgatctggaaaccaccggtctggatccgcataccagccgtccgcgtctgctgtctctggccatgcctggtgcagttgttgtttttgacctgtttggtgttccgctggaagttttttatccgctgtttagccgtgaagaaggtccgctgctggttggtcataatctgaaatttgatctgctgtttctgctgaaagcaggtgtttggcgtgcaagcggtaaacgtctgtgggataccggtctggcccatcaggttctgcatgcacaggcacgtatgcctgcactgaaagatctggctccgggtctggataaaaccctgcagaccagcgattggggtggtccgctgtctagcgaacaggttgcatatgcagcactggatgcagcagttccgctggttctgtatcgtgaacagcgtgaacgtgcacgtaccctgcgtctggaaaaagttctggaagttgaacgtcgtgcactgcctgcagttgcatggatggaactgcgtggtgttccgtttgcaccggaactgtgggaagaagcagcacgcgaagcagaacgtgaagccgaagcactgcgtggtgaactgccgtttggtgttaattggaattctccggcacaggttctggcctatctgaaaggtgaaggtctggatctgccggatacccgtgaagataccctggctggttatcgtgaacatccgctggttgcaaaactgctgcgttatcgcgaagcagcaaaacgtgttagcacctatggtaaagaatgggccaaacatctgaatccggcaaccggtcgtattcatccgagctggcagcagattggtgcagaaaccggtcgcatggcatgtcgtaaaccgaatctgcagcaggttccgcgtgatccggcactgcgtcgtgcatttcgtccgaaagaaggtcgtgttatgctgaaagccgattttagccagattgaactgcgtattgcagcagcaattgcaaaagaaggtcgcatgctgcgcgcctttcgtgaaggtaaagatctgcatgcactgaccgcaagcctggttctgggtaaaccgctggaagaagtgggtaaagaagatcgtcagctggccaaagcactgaattttggtctgctgtatggtctgggtgcagaaggtctgcgtcgttacgccctgaccgcatatggtgttaaactgaccctggaagaagcacagaaactgcgcgatgcattttttcgtgcatatccggctctgaaacgttggcatcgtagccagccggaaggtgaagttgttgttcgtaccctgctgggtcgtcgtcgtaccaccgatcgttataccgaaaaactgaatacaccggttcagggcaccggtgcagatggtctgaaaatggcactggccctgctgtgggaaaatcgtggtctgctgtggggtgcatttccggttctggccgttcatgatgaagttgttctggaagcaccggaagaaggtgcaaaagaatatctggaaaccctgaccgcactgatgcgccagggtatggaagaagttctgggcggcgcagttccggttgaagttgaaggtggtatttatcgtgattggggtgcaacaccgtgggaagaggcctaa

SEQ ID NO: 2 - protein, 564 aa, Artificial Sequence
  Amino acid sequence of Tpol
MASAEGFELHYIPEVGPGMGELLDLLMRQPVLGVDLETTGLDPHTSRPRLLSLAMPGAVVVFDLFGVPLEVFYPLFSREEGPLLVGHNLKFDLLFLLKAGVWRASGKRLWDTGLAHQVLHAQARMPALKDLAPGLDKTLQTSDWGGPLSSEQVAYAALDAAVPLVLYREQRERARTLRLEKVLEVERRALPAVAWMELRGVPFAPELWEEAAREAEREAEALRGELPFGVNWNSPAQVLAYLKGEGLDLPDTREDTLAGYREHPLVAKLLRYREAAKRVSTYGKEWAKHLNPATGRIHPSWQQIGAETGRMACRKPNLQQVPRDPALRRAFRPKEGRVMLKADFSQIELRIAAAIAKEGRMLRAFREGKDLHALTASLVLGKPLEEVGKEDRQLAKALNFGLLYGLGAEGLRRYALTAYGVKLTLEEAQKLRDAFFRAYPALKRWHRSQPEGEVVVRTLLGRRRTTDRYTEKLNTPVQGTGADGLKMALALLWENRGLLWGAFPVLAVHDEVVLEAPEEGAKEYLETLTALMRQGMEEVLGGAVPVEVEGGIYRDWGATPWEEA

SEQ ID NO: 3 - DNA, 1122 bp, Artificial Sequence
  Nucleotide Sequence of Truncated ORF904 Primase
atggctagcgccattaataaacgcagcaaagtgattctgcatggcaatgtgaaaaaaacccgtcgtaccggtgtttatatgattagcctggataatagcggcaataaagattttagcagcaattttagcagcgaacgtattcgctatgcaaaatggtttctggaacatggctttaatattattccgattgatccggaaagcaaaaaaccggttctgaaagaatggcagaaatatagccatgaaatgccgtccgatgaagaaaaacagcgctttctgaaaatgattgaagaaggctataattacgcaattccgggtggtcagaaaggtctggtgattctggattttgaaagcaaagaaaaactgaaagcctggattggtgaaagcgcactggaagaactgtgtcgtaaaaccctgtgtaccaataccgttcatggtggcattcatatttatgttctgagcaatgatattccgccgcataaaattaatccgctgtttgaagaaaatggcaaaggcattattgatctgcagagctataatagctatgttctgggtctgggtagctgtgttaatcatctgcattgcaccaccgataaatgtccgtggaaagaacagaattataccacctgctataccctgtataatgaactgaaagaaattagcaaagtggatctgaaaagcctgctgcgttttctggccgaaaaaggtaaacgtctgggtattacactgagcaaaaccgcaaaagaatggctggaaggcaaaaaagaagaagaagataccgttgttgaatttgaagaactgcgcaaagaactggttaaacgtgatagcggtaaaccggtggaaaaaattaaagaagaaatttgcaccaaaagcccgccgaaactgattaaagaaattatttgcgaaaacaaaacctatgccgatgtgaatattgatcgtagccgtggtgattggcatgttattctgtatctgatgaaacatggtgttaccgatccggataaaattctggaactgctgccgcgtgatagcaaagcaaaagaaaatgaaaaatggaatacccagaaatattttgtgattaccctgagcaaagcatggtctgtggtgaaaaaatatctggaagcctaa

SEQ ID NO: 4 - protein, 373 aa, Artificial Sequence
  Amino Acid Sequence of Truncated ORF904 Primase
MASAINKRSKVILHGNVKKTRRTGVYMISLDNSGNKDFSSNFSSERIRYAKWFLEHGFNIIPIDPESKKPVLKEWQKYSHEMPSDEEKQRFLKMIEEGYNYAIPGGQKGLVILDFESKEKLKAWIGESALEELCRKTLCTNTVHGGIHIYVLSNDIPPHKINPLFEENGKGIIDLQSYNSYVLGLGSCVNHLHCTTDKCPWKEQNYTTCYTLYNELKEISKVDLKSLLRFLAEKGKRLGITLSKTAKEWLEGKKEEEDTVVEFEELRKELVKRDSGKPVEKIKEEICTKSPPKLIKEIICENKTYADVNIDRSRGDWHVILYLMKHGVTDPDKILELLPRDSKAKENEKWNTQKYFVITLSKAWSVVKKYLEA

SEQ ID NO: 5 - DNA, 40 bp, Artificial Sequence
  Oligo M13mp18-R
aacgccagggttttcccagtcacgacgttgtaaaacgacg

SEQ ID NO: 6 - DNA, 17 bp, Artificial Sequence
  M13-20 Primer
gtaaaacgacggccagt

SEQ ID NO: 7 - DNA, 19 bp, Artificial Sequence
  M13 Reverse Primer
ggaaacagctatgaccatg

SEQ ID NO: 8 - DNA, 52 bp, Artificial Sequence
  DnaG-F Primer
attaggtctcagcgcccatcatcatcaccatcatgctggacgaatcccacgc

SEQ ID NO: 9 - DNA, 38 bp, Artificial Sequence
  DnaG-R Primer
ttaaggtctcatatcattactttttcgccagctcctgg

SEQ ID NO: 10 - protein, 590 aa, Escherichia coli
MASAHHHHHHAGRIPRVFINDLLARTDIVDLIDARVKLKKQGKNFHACCPFHNEKTPSFTVNGEKQFYHCFGCGAHGNAIDFLMNYDKLEFVETVEELAAMHNLEVPFEAGSGPSQIERHQRQTLYQLMDGLNTFYQQSLQQPVATSARQYLEKRGLSHEVIARFAIGFAPPGWDNVLKRFGGNPENRQSLIDAGMLVTNDQGRSYDRFRERVMFPIRDKRGRVIGFGGRVLGNDTPKYLNSPETDIFHKGRQLYGLYEAQQDNAEPNRLLVVEGYMDVVALAQYGINYAVASLGTSTTADHIQLLFRATNNVICCYDGDRAGRDAAWRALETALPYMTDGRQLRFMFLPDGEDPDTLVRKEGKEAFEARMEQAMPLSAFLFNSLMPQVDLSTPDGRARLSTLALPLISQVPGETLRIYLRQELGNKLGILDDSQLERLMPKAAESGVSRPVPQLKRTTMRILIGLLVQNPELATLVPPLENLDENKLPGLGLFRELVNTCLSQPGLTTGQLLEHYRGTNNAATLEKLSMWDDIADKNIAEQTFTDSLNHMFDSLLELRQEELIARERTHGLSNEERLELWTLNQELAKK

SEQ ID NO: 11 - DNA, 1749 bp, Phage T7
catcatcatcatcaccacgacaacagccacgatagcgattccgttttcctgtatcacatcccgtgtgacaattgtggttcctcagatggcaatagcctgttctcagacggtcacaccttttgctatgtgtgtgagaaatggaccgccggtaatgaggatacgaaagagcgtgcctctaaacgtaaaccgagtggcgggaaaccaatgacctataatgtgtggaacttcggcgaaagcaatggtcgttattctgccctgactgcccgtgggattagtaaagaaacctgccagaaagcggggtattggatcgctaaagtggatggggtgatgtatcaggttgccgattatcgtgatcagaatgggaacattgtgagtcaaaaagtccgtgacaaagacaaaaacttcaaaacaaccgggagccataaaagtgacgccctgtttggtaaacacctgtggaatgggggtaagaaaatcgtcgtaaccgagggtgaaattgatatgctgacagtaatggagctgcaggactgtaaatatccggtggtatcactgggacatggtgcttcagctgccaagaaaacatgtgccgccaactatgagtatttcgaccagtttgagcaaatcatcctgatgttcgatatggatgaagccggtcgtaaagcagtggaagaagctgcccaggttctgccagctggtaaagttcgtgttgctgtactgccgtgtaaagatgccaatgagtgccacctgaatggtcatgatcgtgagatcatggaacaggtctggaacgctggtccttggatccctgatggtgttgttagcgctctgtcactgcgtgagcgtattcgtgagcatctgtccagcgaagaaagtgttggtctgctgtttagtgggtgtaccggtattaatgacaaaaccctgggtgctcgtgggggtgaagtgattatggtgaccagtggtagcggtatgggtaaaagcacgtttgttcgccagcaagcactgcaatggggtactgctatgggcaagaaagtgggtctggccatgctggaagagtctgtggaggaaaccgccgaggatctgattggactgcataaccgtgtacgcctgcgccaaagcgacagcctgaaacgtgaaatcatcgagaacgggaaatttgatcagtggttcgacgaactgttcgggaatgacacgttccatctgtatgacagctttgccgaggcagaaaccgatcgcctgctggctaaactggcctatatgcgctctgggctgggttgtgacgtgatcatcctggaccatattagcattgtggtgtccgcttcaggagagtcagacgagcgtaaaatgattgataatctgatgaccaaactgaaaggcttcgccaaatcaacgggcgttgtactggtggtaatctgtcacctgaaaaacccggacaaaggcaaagcacacgaagaaggtcgtccggttagtatcaccgatctgcgtggtagtggtgcgctgcgtcaactgagcgatacgattattgctctggagcgtaaccagcaaggggatatgcctaatctggttctggtccgtattctgaaatgccgcttcaccggcgatactggtattgccggctatatggagtataacaaagagactggctggctggaaccgtcatcttatagcggcgaggaggagtctcattcggaaagcacggattggagcaacgatactgatttttgataaagcgctgcactgagctaatgatatgagacc

SEQ ID NO: 12 - DNA, 27 bp, Artificial Sequence
  Heli-K318A-F Primer
gcgtcgacgtttgttcgccagcaagca

SEQ ID NO: 13 - DNA, 21 bp, Artificial Sequence
  Heli-K318A-R Primer
acccataccgctaccactggt

SEQ ID NO: 14 - protein, 575 aa, Artificial Sequence
  Amino Acid Sequence of gp4 K318A
MASAHHHHHHDNSHDSDSVFLYHIPCDNCGSSDGNSLFSDGHTFCYVCEKWTAGNEDTKERASKRKPSGGKPMTYNVWNFGESNGRYSALTARGISKETCQKAGYWIAKVDGVMYQVADYRDQNGNIVSQKVRDKDKNFKTTGSHKSDALFGKHLWNGGKKIVVTEGEIDMLTVMELQDCKYPVVSLGHGASAAKKTCAANYEYFDQFEQIILMFDMDEAGRKAVEEAAQVLPAGKVRVAVLPCKDANECHLNGHDREIMEQVWNAGPWIPDGVVSALSLRERIREHLSSEESVGLLFSGCTGINDKTLGARGGEVIMVTSGSGMGASTFVRQQALQWGTAMGKKVGLAMLEESVEETAEDLIGLHNRVRLRQSDSLKREIIENGKFDQWFDELFGNDTFHLYDSFAEAETDRLLAKLAYMRSGLGCDVIILDHISIVVSASGESDERKMIDNLMTKLKGFAKSTGVVLVVICHLKNPDKGKAHEEGRPVSITDLRGSGALRQLSDTIIALERNQQGDMPNLVLVRILKCRFTGDTGIAGYMEYNKETGWLEPSSYSGEEESHSESTDWSNDTDF

SEQ ID NO: 15 - protein, 280 aa, Artificial Sequence
  Amino Acid Sequence of Truncated T7 gp4A, HeliTrunc
MASAHHHHHHDNSHDSDSVFLYHIPCDNCGSSDGNSLFSDGHTFCYVCEKWTAGNEDTKERASKRKPSGGKPMTYNVWNFGESNGRYSALTARGISKETCQKAGYWIAKVDGVMYQVADYRDQNGNIVSQKVRDKDKNFKTTGSHKSDALFGKHLWNGGKKIVVTEGEIDMLTVMELQDCKYPVVSLGHGASAAKKTCAANYEYFDQFEQIILMFDMDEAGRKAVEEAAQVLPAGKVRVAVLPCKDANECHLNGHDREIMEQVWNAGPWIPDGVVSAALS

(19) United States
(12) Patent Application Publication
(10) Pub. No.: US 2011/0294167 A1
(43) Pub. Date: Dec. 1, 2011

(54) NUCLEIC ACID AMPLIFICATION

(75) Inventors: Paul McEwan, Camps Bay (ZA); Bjarne Faurholm, Rondebosch (ZA); Eric Van Der Walt, Observatory (ZA)

(73) Assignee: KAPABIOSYSTEMS, Woburn, MA (US)

(21) Appl. No.: 13/132,305

(22) PCT Filed: Dec. 2, 2009
(86) PCT No.: PCT/US09/66397

§ 371 Date: Aug. 12, 2011

Related U.S. Application Data
(60) Provisional application No. 61/119,136, filed on Dec. 2, 2008.

(51) Int. Cl. C12P 19/34; C12N 9/12

(52) U.S. Cl. 435/91.2; 435/194

(57) ABSTRACT

The present invention provides improved systems and methods for amplifying nucleic acids. Among other things the present invention provides a system for amplifying nucleic acids through use of a primase and a polymerase with strand-displacement ability without, for example, exogenously-added primers. The present invention is particularly useful for whole genome amplification.

The present application claims priority to U.S. Provisional patent application Ser. No. 61/119,136, filed on Dec. 2, 2008, the entire disclosure of which is incorporated herein by reference.

### BACKGROUND OF THE INVENTION

A requirement for genetic analysis is the availability of sufficient DNA of good quality. For many types of samples, the amount of DNA might be limiting. For example, DNA from human biopsies, blood, forensic samples or single cells is often limited in quantity. Further, DNA from certain samples (e.g., forensic samples) is often partially degraded. Methods for amplifying all the DNA in a sample are generally referred to as methods for Whole-Genome-Amplification (WGA). The aim is to produce more DNA that as closely as possible is a faithful representation of the DNA prior to amplification. The sequence of the amplified DNA is important in some downstream applications (e.g., cloning); hence WGA with high fidelity is useful. The length of amplified DNA is also important when the amplified DNA is to be cloned. Long amplification products enable cloning of long fragments of DNA. A very important quality measure of the amplified DNA is bias. For many applications, in particular copy-number-variation analysis (CNV), it is important that there is minimal bias in the amplification. Bias means that some part(s) of the DNA is amplified in preference to other parts. It is important that each part (locus) of the genome is amplified to the same extent.

Several methods have been developed for WGA. These methods generally involve either PCR-amplification or isothermal amplification. PCR-dependent WGA methods include PEP-PCR, DOP-PCR and ligation-mediated PCR (LMP). In PEP-PCR, 15-base random oligonucleotides are used as primers (Zhang et al., 1992, Proc. Natl. Acad. Sci. 89:5847-5851). Annealing takes place at a low temperature to enable annealing throughout the genome. DOP-PCR employs semi-degenerate primers. The middle part of the primer is degenerate flanked by non-degenerate nucleotides Annealing is done at a low temperature in the first few cycles followed by cycles with a higher annealing temperature (Telenius et al., 1992, Genomics 13:718-725). Both PEP-PCR and DOP-PCR generally use Taq polymerase and the resulting PCR products are mostly less than 3 kb. Both PEP-PCR and DOP-PCR have a large amplification biases (Pinard et al., 2006, BMC Genomics 7:216). LMP utilizes fragmented DNA to which linkers are ligated followed by PCR amplification with universal primers. (US Publication No. 20040209299). A variation of this method involves using semi-random primers in which the 3′ part of the oligonucleotide is random and the 5′ part provides binding sites for a universal primer. In the initial step the semi-random oligonucleotide anneals to various places in the genome. This is followed by a PCR with the universal primer to generate the amplified library. As in PEP-PCR and DOP-PCR, the amplicon length is generally less than 3 kb. The fidelity is limited to the fidelity of the polymerase used, generally Taq polymerase. The bias depends on the ability of the polymerase to read through areas that are difficult to amplify. Areas rich in GC or AT may amplify less during the PCR leading to a large bias in the amplified product.

Isothermal WGA methods include T7-based linear amplification of DNA (TLAD), multiple displacement amplification (MDA) and helicase-dependent amplification (HDA). In TLAD poly-T tails are added to DNA fragments using terminal transferase. A primer with having poly-A at the 3′ end and a T7 promoter at the 5′ end is annealed to the DNA. Klenow is used to extend the primer forming dsDNA fragments with a T7 promoter at one end. T7 RNA polymerase is used to transcribe the DNA producing large amounts of RNA linearly amplified from the adaptor-modified DNA (Liu et al., 2003, BMC Genomics 4(1):19). The product of this amplification method is RNA which will mostly require reverse-transcription prior to down-stream analysis. The method also appears cumbersome in that many steps are involved.

In MDA, the template DNA is typically denatured in the presence of short random primers, e.g., hexamers. The primers are then extended by a strand-displacing enzyme, e.g., Phi29 DNA polymerase or Bst DNA polymerase. Primers bind several places on the template DNA strand and extension may occur from several annealed primers on the same template strand. The polymerase, due to its strong strand-displacement activity, will then displace newly replicated strands. Random primers will bind to the displaced strands that will now become template for replication (U.S. Pat. No. 6,977,148, U.S. Pat. No. 6,617,137, U.S. Pat. No. 6,280,949, U.S. Pat. No. 6,642,034). Due to the use of random primers, background amplification can be produced.

HDA typically utilizes a set of replication enzymes from phage T7, which basically reconstitute the T7 replication complex in vitro (see, US Publication No. 20050164213). HDA has been further modified by Li and co-workers (Li et al., 2008, Nucleic Acids Research 36(13):e79). However, this system is highly complex and involves the use of a multi-protein system including the T7 gp4 helicase/primase enzyme, the T7 gp2.5 ssDNA binding protein, T7 polymerase, T7 sequenase, nucleotide diphosphokinase, pyrophosphatase, and creatine kinase. For example, in the HDA amplification system, DNA is unwound by the helicase part of T7 gp4. The primase part of gp4 synthesizes primers on the ssDNA and the primers are extended by a blend of mutant T7 DNA polymerase which lacks the 3′ to 5′ exonuclease activity and wild-type T7 DNA polymerase. The method further makes use of T7 gp2.5—a single-stranded DNA binding protein to stabilize ssDNA and a pyrophosphatase to eliminate inhibition by pyrophosphate. The helicase activity of T7 gp4 requires hydrolysis of dTTP or ATP. The method of Li et al. includes creatine kinase and creatine phosphate to generate ATP and nucleotide diphosphokinase to phosphorylate dTDP to dTTP. The fidelity of amplified product is typically low due to the use of exo-polymerase as the main component of the polymerase blend.

Therefore, there is a need for more effective and less biased whole genome amplification methods.

### SUMMARY OF THE INVENTION

The present invention provides improved systems and methods for amplifying nucleic acids including whole genome nucleic acids. Among other things, the present invention provides a simplified system for effectively and accurately amplifying nucleic acids through use of a primase and a polymerase with strand-displacement ability.

In one aspect, the present invention provides methods for amplifying nucleic acids comprising a step of incubating a template nucleic acid and an amplification mixture comprising a primase and a polymerase having strand-displacement ability such that the template nucleic acid becomes amplified. In some embodiments, the amplification mixture does not contain exogenously-added oligonucleotide primers. In some embodiments, the amplification mixture does not contain a helicase. In some embodiments, the amplification mixture does not contain ssDNA binding proteins. In some embodiments, the amplification mixture does not contain an ATP regeneration system.

In certain embodiments, the template nucleic acid comprises genomic DNA. In some embodiments, the genomic DNA comprises an entire genome. In some embodiments, the genomic DNA is human DNA. In some embodiments, the template nucleic acid is obtained from a human biopsy, blood, a forensic sample, and/or a single cell.

In some embodiments, the template nucleic acid is RNA. In some such embodiments, inventive methods of the invention further include a step of generating a cDNA using a reverse transcriptase.

In some embodiments, the template nucleic acid and the amplification mixture are incubated at a substantially constant temperature. In some embodiments, the template nucleic acid and the amplification mixture are incubated with a thermal cycling program.

In some embodiments, the primase is selected from the group consisting of ORF904 primase, a primase from Solfolobus solfataricus, p41-p46 primase complex from Pyrococcus furiosus, a primase from Pyrococcus horikoshii, phage T7 primase (e.g., phage T7 helicase-deficient primase), E. coli dnaG primase, and fragments thereof.

In some embodiments, the polymerase is selected from the group consisting of Phi29 polymerase, Pyrophage 3173 or exonuclease minus version thereof, KOD polymerase, Vent or DeepVent polymerases, Bst polymerase, KapaHiFi™ DNA polymerase and combination thereof. In some embodiments, the polymerase is hyperthermophilic. In some embodiments, the polymerase is thermostable.

In some embodiments, the amplification mixture further comprises one or more low-temperature melting reagents (e.g., betaine, DMSO, or glycerol). In some embodiments, the amplification mixture further comprises a thermoprotectant (e.g., ectoine, hydroxy ectoine, mannosylglycerate, trehalose, betaine, glycerol or proline).

In another aspect, the present invention provides compositions for amplifying nucleic acid according to various methods described herein. In some embodiments, inventive compositions according to the invention contain a primase, a polymerase having strand-displacement ability, and template nucleic acid (e.g., genomic DNA such as an entire genome), wherein the composition does not contain exogenously-added oligonucleotide primers as described herein. In some embodiments, inventive compositions according to the invention do not contain a helicase. In some embodiments, inventive compositions according to the invention do not contain ssDNA binding proteins. In some embodiments, inventive compositions according to the invention do not contain an ATP regeneration system. In some embodiments, inventive compositions of the invention do not contain any of helicase, ssDNA binding proteins, or enzymes for ATP generation.

In yet another aspect, the present invention provides methods and compositions for amplifying nucleic acids (e.g., genomic DNA such as an entire genome) using an amplification system containing less than 7 (e.g., less than 6, 5, 4, 3, 2) proteins or enzymes without exogenously-added oligonucleotide primers. In some embodiments, inventive methods and compositions according to the invention utilize a two-protein system to amplify nucleic acids (e.g., genomic DNA such as an entire genome). In some embodiments, the two-protein system contains a primase and a polymerase with strand-displacement ability.

In this application, the use of “or” means “and/or” unless stated otherwise. As used in this application, the term “comprise” and variations of the term, such as “comprising” and “comprises,” are not intended to exclude other additives, components, integers or steps. As used herein, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWINGS

The drawings are for illustration purposes only not for limitation.

FIG. 1 depicts an exemplary DNA amplification with ORF904 primase with Taq or Tpol polymerases. Lane 1: no primer/primase or polymerase added; lane 2: no primer/primase with 0.1 U Taq pol; lane 3: no primer/primase with 5 ng Tpol; lane 4: no primer/primase with 0.5 ng Tpol; lane 5: primer with no polymerase; lane 6: primer with 0.1 U Taq; lane 7: primer with 5 ng Tpol, lane 8: primer with 0.5 ng Tpol; lane 9: 34 ng ORF904 primase with no polymerase; lane 10: 34 ng ORF904 primase with 0.1 U Taq; lane 11: 34 ng ORF904 primase with 5 ng Tpol; lane 12: 34 ng ORF904 primase with 0.5 ng Tpol; lane 13: 3.4 ng ORF904 primase with no polymerase; lane 14: 3.4 ng ORF904 primase with 0.1 U Taq; lane 15: 3.4 ng ORF904 primase with 5 ng Tpol; lane 16: 3.4 ng ORF904 primase with 0.5 ng Tpol.

FIG. 2 depicts an exemplary DNA amplification with Phi29 polymerase with ORF904 primase. Lane 1: No primer/primase, 100 ng Phi29; 2) 50 uM random hexamer, 100 ng Phi29; 3) 5 uM random hexamer, 100 ng Phi29; 4) 50 ng ORF904 primase, no Phi29; 5) 150 ng ORF904 primase, no Phi29; 6) 500 ng ORF904 primase, no Phi29; 7) 1500 ng ORF904 primase, no Phi29; 8) 50 ng ORF904 primase, 100 ng Phi29; 9) 150 ng ORF904 primase, 100 ng Phi29; 10) 500 ng ORF904 primase, 100 ng Phi29; 11) 1500 ng ORF904 primase, 100 ng Phi29.

FIG. 3 depicts an exemplary amplification of M13 and lambda DNA with ORF904 primase and Bst DNA polymerase. Lanes 1-7: M13 DNA as template, lanes 8-14: lambda DNA as template. Lanes 1 and 8, random hexamer; lanes 2 and 9, 1500 ng primase, no polymerase; lanes 3 and 10, 750 ng primase, no polymerase, lanes 4 and 11, 500 ng primase, no polymerase; lanes 5 and 12, 1500 ng primase and 8 U Bst polymerase; lanes 6 and 13, 750 ng primase and 8 U Bst polymerase; lanes 7 and 14, 500 ng primase and 8 U Bst polymerase.

FIG. 4 depicts an exemplary amplification of M13 DNA with ORF904 primase and Bst DNA polymerase. Lane 1, Bst polymerase, no primer or primase; lane 2, no polymerase, 20 μM random hexamer; lane 3, Bst polymerase, 20 μM random hexamer; lane 4, no polymerase, 500 ng primase; lane 5, Bst polymerase, 500 ng primase; lane 6, Bst polymerase, 50 ng primase; lane 7, Bst polymerase, 500 ng primase, 0.1 mM NTPs.

FIG. 5 depicts an exemplary amplification of M13 DNA with gp4 K318A, Phi29 and T7 DNA polymerase.

FIG. 6 depicts an exemplary restriction digest of amplified M13 DNA. Marker: GeneRuler, Fermentas. Lanes 1-3 are MboI-digested amplification products of reactions 6-8, example 9.

FIG. 7 depicts an exemplary amplification of human genomic DNA with gp4 K318A, Phi29 and T7 DNA polymerase.

### DEFINITIONS

In order for the present invention to be more readily understood, certain terms are first defined below. Additional definitions for the following terms and other terms are set forth throughout the specification.

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, and/or substitution with other chemical without adversely affecting their activity. Amino acids may participate in a disulfide bond. The term “amino acid” is used interchangeably with “amino acid residue,” and may refer to a free amino acid and/or to an amino acid residue of a peptide. It will be apparent from the context in which the term is used whether it refers to a free amino acid or a residue of a peptide. It should be noted that all amino acid residue sequences are represented herein by formulae whose left and right orientation is in the conventional direction of amino-terminus to carboxy-terminus.

Base Pair (bp): As used herein, base pair refers to a partnership of adenine (A) with thymine (T), or of cytosine (C) with guanine (G) in a double stranded DNA molecule.

Complementary: As used herein, the term “complementary” refers to the broad concept of sequence complementarity between regions of two polynucleotide strands or between two nucleotides through base-pairing. It is known that an adenine nucleotide is capable of forming specific hydrogen bonds (“base pairing”) with a nucleotide which is thymine or uracil. Similarly, it is known that a cytosine nucleotide is capable of base pairing with a guanine nucleotide.

Constant temperature: As used herein, the term “constant temperature,” when used in the context of nucleic acid amplification, refers to an amplification reaction that is carried out under isothermal conditions as opposed to thermocycling conditions. Typically, thermocycling conditions are used by polymerase chain reaction methods in order to denature the DNA and anneal new primers after each cycle. Constant temperature procedures rely on other methods to denature the DNA, such as the strand displacement ability of some polymerases or of DNA helicases that act as accessory proteins for some DNA polymerases. Thus, the term “constant temperature” does not mean that no temperature fluctuation occurs, but rather indicates that the temperature variation during the amplification process is not sufficiently great to provide the predominant mechanism to denature product/template hybrids. In some embodiments, a constant temperature for nucleic acid amplification is at or less than 60° C. (e.g., at or less than 50° C., 45° C., 40° C., 35° C., 30° C., 25° C., 20° C.).

Fidelity: As used herein, the term “fidelity” refers to the accuracy of DNA polymerization by template-dependent DNA polymerase. The fidelity of a DNA polymerase is typically measured by the error rate (the frequency of incorporating an inaccurate nucleotide, i.e., a nucleotide that is not complementary to the template nucleotide). The accuracy or fidelity of DNA polymerization is maintained by both the polymerase activity and the 3′-5′ exonuclease activity of a DNA polymerase. The term “high fidelity” refers to an error rate less than 4.45×10⁻⁶ (e.g., less than 4.0×10⁻⁶, 3.5×10⁻⁶, 3.0×10⁻⁶, 2.5×10⁻⁶, 2.0×10⁻⁶, 1.5×10⁻⁶, 1.0×10⁻⁶, 0.5×10⁻⁶) mutations/nt/doubling. The fidelity or error rate of a DNA polymerase may be measured using assays known to the art. For example, the error rates of DNA polymerases can be tested using the lad PCR fidelity assay described in Cline, J. et al. (Cline, et al., 1996, Nucleic Acids Research 24: 3546-3551). Briefly, a 1.9 kb fragment encoding the lacIOlacZa target gene is amplified from pPRIAZ plasmid DNA using 2.5 U DNA polymerase (i.e., amount of enzyme necessary to incorporate 25 nmoles of total dNTPs in 30 min. at 72° C.) in the appropriate PCR buffer. The lacI-containing PCR products are then cloned into lambda GT10 arms, and the percentage of lacI mutants (MF, mutation frequency) is determined in a color screening assay, as described (Lundberg, K. S., et al., 1991 Gene 180: 1-8). Error rates are expressed as mutation frequency per by per duplication (MF/bp/d), where by is the number of detectable sites in the lad gene sequence (349) and d is the number of effective target doublings. Similar to the above, any plasmid containing the lacIOlacZa target gene can be used as template for the PCR. The PCR product may be cloned into a vector different from lambda GT (e.g., plasmid) that allows for blue/white color screening.

Functional variants: As used herein, the term “functional variants” denotes, in the context of a functional variant of an amino acid sequence, a molecule that retains a biological activity (e.g., primase or polymerase activity) that is substantially similar to that of the original sequence. A functional variant or equivalent may be a natural derivative or is prepared synthetically. Exemplary functional variants include amino acid sequences having substitutions, deletions, or additions of one or more amino acids, provided that the biological activity of the original protein is conserved (e.g., primase or polymerase activity). For example, a functional variant may have an amino acid sequence at least 70%, 75%, 80%, 85%, 90%, 95%, 96%, 97%, 98%, 99% identical to the amino acid sequence of an original protein (e.g., a primase or polymerase).

Helicase: As used herein, the term “helicase” refers to a class of enzymes that typically are motor proteins that move directionally along a nucleic acid backbone, separating two annealed nucleic acid strands (i.e., DNA, RNA, or RNA-DNA hybrid) using energy derived from ATP hydrolysis or other sources.

In vitro: As used herein, the term “in vitro” refers to events that occur in an artificial environment, e.g., in a test tube or reaction vessel, in cell culture, etc., rather than within a multi-cellular organism.

Mutation: As used herein, the term “mutation” refers to a change introduced into a parental sequence, including, but not limited to, substitutions, insertions, deletions (including truncations). The consequences of a mutation include, but are not limited to, the creation of a new character, property, function, phenotype or trait not found in the protein encoded by the parental sequence.

Mutant: As used herein, the term “mutant” refers to a modified protein which displays altered characteristics when compared to the parental protein.

Joined: As used herein, “joined” refers to any method known in the art for functionally connecting polypeptide domains, including without limitation recombinant fusion with or without intervening domains, inter-mediated fusion, non-covalent association, and covalent bonding, including disulfide bonding, hydrogen bonding, electrostatic bonding, and conformational bonding.

Nucleotide: As used herein, a monomeric unit of DNA or RNA consisting of a sugar moiety (pentose), a phosphate, and a nitrogenous heterocyclic base. The base is linked to the sugar moiety via the glycosidic carbon (1′ carbon of the pentose) and that combination of base and sugar is a nucleoside. When the nucleoside contains a phosphate group bonded to the 3′ or 5′ position of the pentose it is referred to as a nucleotide. A sequence of operatively linked nucleotides is typically referred to herein as a “base sequence” or “nucleotide sequence,” and is represented herein by a formula whose left to right orientation is in the conventional direction of 5′-terminus to 3′-terminus.

Oligonucleotide or Polynucleotide: As used herein, the term “oligonucleotide” is defined as a molecule including two or more deoxyribonucleotides and/or ribonucleotides, preferably more than three. Its exact size will depend on many factors, which in turn depend on the ultimate function or use of the oligonucleotide. The oligonucleotide may be derived synthetically or by cloning. As used herein, the term “polynucleotide” refers to a polymer molecule composed of nucleotide monomers covalently bonded in a chain. DNA (deoxyribonucleic acid) and RNA (ribonucleic acid) are examples of polynucleotides.

Polymerase: As used herein, a “polymerase” refers to an enzyme that catalyzes the polymerization of nucleotide (i.e., the polymerase activity). Generally, the enzyme will initiate synthesis at the 3′-end of the primer annealed to a polynucleotide template sequence, and will proceed toward the 5′ end of the template strand. A “DNA polymerase” catalyzes the polymerization of deoxynucleotides.

Primase: As used herein, the term “primase” refers to an enzyme with primase activity, i.e., the ability to synthesize small RNA or DNA segments (called primers). Typically, a primase uses a single-strand DNA (ssDNA) as template. The primase may bind the DNA template and provide at least one initial nucleotide from which a DNA polymerase can catalyze the addition of nucleotides complementary to the DNA template. Primases can also have additional enzymatic activities, including, for example, DNA helicase and polymerase activity.

Primer: As used herein, the term “primer” refers to an oligonucleotide, whether occurring naturally or produced synthetically, which is capable of acting as a point of initiation of nucleic acid synthesis when placed under conditions in which synthesis of a primer extension product which is complementary to a nucleic acid strand is induced, e.g., in the presence of four different nucleotide triphosphates and polymerase in an appropriate buffer (“buffer” includes pH, ionic strength, cofactors, etc.) and at a suitable temperature. The primer is preferably single-stranded for maximum efficiency in amplification, but may alternatively be double-stranded. If double-stranded, the primer is first treated to separate its strands before being used to prepare extension products. Preferably, the primer is an oligodeoxyribonucleotide. The primer must be sufficiently long to prime the synthesis of extension products in the presence of the polymerase. The exact lengths of the primers will depend on many factors, including temperature, source of primer and use of the method. For example, depending on the complexity of the target sequence, the oligonucleotide primer typically contains 15-25 nucleotides, although it may contain more or few nucleotides. Short primer molecules generally require colder temperatures to form sufficiently stable hybrid complexes with template.

Processivity: As used herein, “processivity” refers to the ability of a polymerase to remain attached to the template and perform multiple modification reactions. “Modification reactions” include but are not limited to polymerization, and exonucleolytic cleavage. In some embodiments, “processivity” refers to the ability of a DNA polymerase to perform a sequence of polymerization steps without intervening dissociation of the enzyme from the growing DNA chains. Typically, “processivity” of a DNA polymerase is measured by the length of nucleotides (for example 20 nts, 300 nts, 0.5-1 kb, or more) that are polymerized or modified without intervening dissociation of the DNA polymerase from the growing DNA chain. “Processivity” can depend on the nature of the polymerase, the sequence of a DNA template, and reaction conditions, for example, salt concentration, temperature or the presence of specific proteins. As used herein, the term “high processivity” refers to a processivity higher than 20 nts (e.g., higher than 40 nts, 60 nts, 80 nts, 100 nts, 120 nts, 140 nts, 160 nts, 180 nts, 200 nts, 220 nts, 240 nts, 260 nts,280 nts, 300 nts, 320 nts, 340 nts, 360 nts, 380 nts, 400 nts, or higher) per association/disassociation with the template. Processivity can be measured according the methods defined herein and in WO 01/92501 A1. In some embodiments, a DNA polymerase with high processivity may generate DNA fragments up to 5 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb or more in length.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Strand Displacement Activity: As used herein, the term “strand displacement activity” refers to an activity of a polymerase that can synthesize DNA by unwinding template without a helicase activity.

Synthesis: As used herein, the term “synthesis” refers to any in vitro method for making new strand of polynucleotide or elongating existing polynucleotide (i.e., DNA or RNA) in a template dependent manner. Synthesis, according to the invention, includes amplification, which increases the number of copies of a polynucleotide template sequence with the use of a polymerase. Polynucleotide synthesis (e.g., amplification) results in the incorporation of nucleotides into a polynucleotide (i.e., a primer), thereby forming a new polynucleotide molecule complementary to the polynucleotide template. The formed polynucleotide molecule and its template can be used as templates to synthesize additional polynucleotide molecules. “DNA synthesis,” as used herein, includes, but is not limited to, PCR, the labeling of polynucleotide (i.e., for probes and oligonucleotide primers), polynucleotide sequencing.

Template DNA molecule: As used herein, the term “template DNA molecule” refers to a strand of a nucleic acid from which a complementary nucleic acid strand is synthesized by a DNA polymerase, for example, in a primer extension reaction.

Template-dependent manner: As used herein, the term “template-dependent manner” refers to a process that involves the template dependent extension of a primer molecule (e.g., DNA synthesis by DNA polymerase). The term “template-dependent manner” typically refers to polynucleotide synthesis of RNA or DNA wherein the sequence of the newly synthesized strand of polynucleotide is dictated by the well-known rules of complementary base pairing (see, for example, Watson, J. D. et al., In: Molecular Biology of the Gene, 4th ed., W. A. Benjamin, Inc., Menlo Park, Calif. (1987)).

Thermocycling conditions: As used herein, the term “thermocycling conditions,” when used in the context of nucleic acid amplification, refers to amplification conditions under which the denaturation of template DNA, annealing of new primers and synthesis of new DNA are carried out at different temperatures.

Thermostable enzyme: As used herein, the term “thermostable enzyme” refers to an enzyme which is stable to heat (also referred to as heat-resistant) and catalyzes (facilitates) polymerization of nucleotides to form primer extension products that are complementary to a polynucleotide template sequence. Typically, thermostable stable polymerases are preferred in a thermocycling process wherein double stranded nucleic acids are denatured by exposure to a high temperature (e.g., about 95 C) during the PCR cycle. A thermostable enzyme described herein effective for a PCR amplification reaction satisfies at least one criteria, i.e., the enzyme do not become irreversibly denatured (inactivated) when subjected to the elevated temperatures for the time necessary to effect denaturation of double-stranded nucleic acids. Irreversible denaturation for purposes herein refers to permanent and complete loss of enzymatic activity. The heating conditions necessary for denaturation will depend, e.g., on the buffer salt concentration and the length and nucleotide composition of the nucleic acids being denatured, but typically range from about 90° C. to about 96° C. for a time depending mainly on the temperature and the nucleic acid length, typically about 0.5 to four minutes. Higher temperatures may be desired as the buffer salt concentration and/or GC composition of the nucleic acid is increased. In some embodiments, thermostable enzymes will not become irreversibly denatured at about 90° C. -100° C. Typically, a thermostable enzyme suitable for the invention has an optimum temperature at which it functions that is higher than about 40° C., which is the temperature below which hybridization of primer to template is promoted, although, depending on (1) magnesium and salt, concentrations and (2) composition and length of primer, hybridization can occur at higher temperature (e.g., 45° C.-70° C.). The higher the temperature optimum for the enzyme, the greater the specificity and/or selectivity of the primer-directed extension process. However, enzymes that are active below 40° C. (e.g., at 30-37° C.) are also within the scope of this invention. In some embodiments, the optimum temperature ranges from about 50° C. to 90° C. (e.g., 60° C.-80° C.).

Whole Genome Amplification: As used herein, the term “whole genome amplification” refers to a method for amplifying all the DNA in a sample. Typically, whole genome amplification refers to amplification of an entire genome in a sample.

Wild-type: As used herein, the term “wild-type” refers to a gene or gene product which has the characteristics of that gene or gene product when isolated from a naturally-occurring source.

### DETAILED DESCRIPTION OF THE INVENTION

The present invention encompasses unexpected discovery that nucleic acid such as a whole genome can be effectively amplified using a simple two-enzyme system, i.e., a primase and a strand-displacing DNA polymerase, without exogenously-added primers. It is contemplated that in the present invention DNA unwinding is accomplished by using strand-displacing polymerases and does not require additional accessory proteins such as helicase, ssDNA binding proteins and/or an ATP regeneration system. Thus, the present invention provides, among other things, systems and methods for amplifying nucleic acids, in particular, genomic DNA such as an entire genome, using a primase and a polymerase with strand-displacement activity without exogenously-added oligonucleotide primers. In some embodiments, inventive systems and methods according to the present invention does not include a helicase, ssDNA binding proteins, an ATP regeneration system, and/or other accessory proteins. In some embodiments, inventive systems and methods according to the present invention contain less than 7 (e.g., less than 6, 5, 4, 3, or 2) proteins or enzymes without exogenously-added oligonucleotide primers. In some embodiments, inventive systems and methods according to the present invention contain two proteins, i.e., a primase and a strand-displacing DNA polymerase. In some embodiments, inventive systems and methods according to the present invention contain one protein with primase and strand-displacing polymerase activities.

Thus, the present invention provides a highly effective, simplified and accurate nucleic acid amplification system. One of many advantages of the present invention is that the amplification systems and methods described herein may provide more even representation of the genome as strand-displacing DNA polymerases allow more complete DNA unwinding as compared to helicase dependent unwinding. Additionally, primases such as the ORF904 primase have very short (e.g., 3 bp) recognition sequences providing dense priming site distribution across genomes. Therefore, the present invention provides methods for amplifying genomes with low amplification bias.

Various aspects of the invention are described in detail in the following sections. The use of sections is not meant to limit the invention. Each section can apply to any aspect of the invention. In this application, the use of “or” means “and/or” unless stated otherwise.

### Nucleic Acid Templates

The present invention may be used to amplify any desired target nucleic acid molecule and does not require that a template nucleic acid have any particular sequence or length. For example, template nucleic acids which may be amplified include any naturally occurring prokaryotic (for example, pathogenic or non-pathogenic bacteria, Escherichia, Salmonella, Clostridium, Agrobacter, Staphylococcus and Streptomyces, Streptococcus, Rickettsiae, Chlamydia, Mycoplasma, etc.), eukaryotic (for example, protozoans and parasites, fungi, yeast, higher plants, lower and higher animals, including mammals and humans) or viral (for example, Herpes viruses, HIV, influenza virus, Epstein-Barr virus, hepatitis virus, polio virus, etc.) or viroid nucleic acid. Template nucleic acid can also be recombinantly generated (e.g., a plasmid) or chemically synthesized. Thus, a template nucleic acid sequence need not be found in nature.

In some embodiments, template nucleic acid can be obtained from tissues, biopsy samples, bodily fluids (for example, blood, serum, stool, plasma, saliva, urine, tears, semen, vaginal secretions, lymph fluid, cerebrospinal fluid or mucosa secretions), forensic samples, fecal matter, individual or a population of cells or extracts thereof, and subcellular structures such as mitochondria or chloroplasts, or inorganic samples, among others. Template nucleic acid can be any nucleic acid, e.g., genomic, plasmid, cosmid, yeast artificial chromosomes, artificial or man-made DNA. In some embodiments, template nucleic acids include all the nucleic acid in a sample. In some embodiments, such template nucleic acids include heterologous nucleic acids including, for example, both human and bacterial, viral or other pathogenic nucleic acid. In some embodiments, template nucleic acids include homologous nucleic acids. For example, template nucleic acids is an entire genome. In some embodiments, template nucleic acid is obtained from a human or animal to be screened for the presence of one or more genetic sequences that can be diagnostic for, or predispose the subject to, a medical condition or disease.

In some embodiments, template nucleic acid is RNA. In some embodiments, RNA template is first converted into cDNA using a reverse transcriptase. Single-stranded RNA, double-stranded RNA or mRNA are also able to be amplified by systems and methods of the invention. For example, the RNA genomes of certain viruses can be converted to DNA by reaction with enzymes such as reverse transcriptase (Maniatis, T. et al., Molecular Cloning (A Laboratory Manual), Cold Spring Harbor Laboratory, 1982; Noonan, K. F. et al., 1988 Nucleic Acids Res. 16:10366). The product of the reverse transcriptase reaction (i.e., cDNA) may then be amplified according to the invention.

### Primases

Primases suitable for the invention may include any enzymes that have primase activity. For example, suitable primases may include those primases that utilize ribonucleotides for RNA primer synthesis, those that utilize deoxyribonucleotides for DNA primer synthesis and those that use both ribonucleotides and deoxyribonucleotides for primer synthesis. In some embodiments, suitable primases include DNA-dependent RNA polymerases that synthesize RNA primers in eukaryotes and bacteria. Exemplary primases include, but are not limited to, primases from Solfolobus solfataricus (Lao-Sirieix, et al., 2004, J. Mol. Biol. 344:1251-1263, incorporated herein by reference), ORF904 primase from the pRN1 plasmid of Solfolobus islandicus (Beck, et al., 2007 Nucleic Acids Research 17:5635-5645, incorporated herein by reference), p41-p46 primase complex from Pyrococcus furiosus (Liu, et al., 2001, Journal of Biological Chemistry 48:45484-45490, incorporated herein by reference), the primase from Pyrococcus horikoshii (Matsui, et al., 2003, Biochemistry 42:14968-14976, incorporated herein by reference), phage T7 primase (e.g., gene 4 protein of phage T7) (US Patent Application 20050164213, incorporated herein by reference), E. coli dnaG primase (acc. no. NC_{—}010473, incorporated herein by reference), gene 41 and 61 of phage T4 (see, e.g., Kornberg and Baker, 1992, DNA Replication, Freeman and Co., New York, supra., incorporated herein by reference). Primases suitable for the invention include fragments or variants of naturally-occurring primases such as those described in Frick, D. N. et al., 1998 Proc. Natl. Acad. Sci. 95:7957-7962, the disclosure of which is hereby incorporated by reference.

Without wishing to be bound by any theory, it is contemplated that, during amplification, synthesis of the lagging strand is initiated from short oligoribonucleotide primers that are synthesized at various sites by primases. Specific interactions between a primase and the DNA polymerase allow the DNA polymerase to initiate DNA synthesis from the oligoribonucleotide resulting in the synthesis of the lagging strand. In general, primases recognize initiation sites along a template nucleic acid. In some embodiments, primases suitable for the present invention recognize at least a di-nucleotide initiation site. In some embodiments, primases suitable for the invention recognizes a three-nucleotide initiation site. In some embodiments, primases suitable for the invention recognize an initiation site containing more than three nucleotides (e.g., 4, 5, 6, 9, 12, 15, 18, 21 or more nucleotides). Typically, primases synthesize primers up to 14 nucleotides long. In some embodiments, primases synthesize primers that are 1, 2, 3, 4, 5, 6, 7, 8, 9, 10, 11, 12, 13, 14 or more nucleotides long.

In some embodiments, a suitable primase for the invention may also have other activity such as helicase activity or polymerase activity. For example, the full length, wild type ORF904 enzyme has both helicase and primase activity (Lipps, et al. 2003, EMBO, 22(10):2516-2525). This thermostable primase was identified on a plasmid from Sulfolobus islandicus. The primase initiates primer synthesis at a tri-nucleotide GTG recognition motif. It utilizes primarily dNTPs for primer synthesis and it is thought that it requires at least one ribonucleotide for primer synthesis. Generally, the primers synthesized by ORF904 are approximately 8 nucleotides long and can be further extended by the primase or heterogeneously added DNA polymerases (e.g., a polymerase with strand-displacement activity or Taq DNA polymerase). The full Open Reading Frame (ORF) of ORF904 encodes a protein with 904 amino acids in which part of the N-terminal domain has homology to primases and polymerases and the C-terminal domain has homology to helicases. As described in the Examples section, truncations of ORF904 including the N-terminal portion (e.g., amino acids 1-370 as shown in SEQ ID NO:4) can be used as primases in nucleic acid amplification methods according to the present invention. It is also contemplated that functional variants based on the N-terminal portion of ORF904 (e.g., amino acids 1-370 as shown in SEQ ID NO:4) can be used as primases in nucleic acid amplification methods according to the present invention. For example, suitable functional variants typically have an amino acid sequence at least 70%, 75%, 80%, 85%, 90%, 95%, 96%, 97%, 98%, 99% identical to SEQ ID NO:4.

Another non-limiting example is the gene 4 protein of the T7 replication system which has both primase and helicase activity (Bernstein and Richardson, 1988 Proc. Natl. Acad. USA 85:396; Bernstein and Richardson, 1989 J. Biol. Chem. 264:13066; Frick, D. N., et al., 1998 Proc. Natl. Acad. Sci. 95:7957-7962, the disclosures of all of which are hereby incorporated by reference). Typically, only the 63-kDa form of the gene 4 protein has primase activity, which typically recognizes specific pentanucleotide initiation sites and synthesizes tetraribonucleotides that are used as primers by T7 DNA polymerase for DNA synthesis. Without wishing to be bound by theory, it is thought that the helicase domains of the phage T7 gp4 protein assemble to form a hexameric ring-shaped structure. One of the ssDNA strands is threaded through the hole of the ring-shaped structure during helicase-dependent dissociation of the two strands of dsDNA. It is thought that this threading activity causes six primase domains to be in close proximity to one another and to the ssDNA. Without wishing to be bound by theory, it is thought that adjacent primase units are important for activity and that the helicase domain essentially acts as a scaffold for bringing primase molecules into close proximity of each other. The T7 helicase utilizes dTTP as energy source for translocation along DNA. As described in the Examples section, mutations at positions such as 318 (e.g., K318A) may abolish helicase activity but only mildly affect the primase activity of gp4. Such helicase-deficient mutant of T7 gp4 protein can be used in nucleic acid amplification reactions according to the invention. The amino acid sequence of an exemplary helicase-deficient T7 gp4 K318A primase is shown in SEQ ID NO:14 (see, Example 8). Functional variants having an amino acid sequence at least 70%, 75%, 80%, 85%, 90%, 95%, 96%, 97%, 98%, 99% identical to SEQ ID NO:14 can also be used in the present invention.

Prokaryotic primases (e.g., primases from bacteria and their phages) are typically single subunit enzymes that possess a zinc-binding motif in the N-terminal domain of the protein and an RNA polymerase domain in the C-terminal region. Primases from archaea and eukaryotes typically are more complex. It is thought that these organisms have primases containing a small catalytic subunit that associates with a larger subunit, which in turn together associate with two additional components to form a primosome complex. For a review of DNA primases, see Frick and Richardson, 2001 Annu. Rev. Biochem, 70:39-80, the contents of which are herein incorporated by reference.

It is contemplated that the oligoribonucleotide primers that are synthesized by primases decrease or eliminate the need for exogenous oligonucleotide primers for nucleic acid amplification according to the invention. In some embodiments, amplification of nucleic acid such as an entire genome according to the invention does not require exogenously-added oligonucleotide primers.

### DNA Polymerase

In general, a polymerase suitable for the present invention can be any polymerase having strand-displacement activity. Suitable polymerases for the present invention may have varying levels of thermophilicity and/or thermostability. In some embodiments, suitable polymerases are hyperthermophilic and/or thermostable, in particular, when the amplification is carried out under thermocycling conditions. Suitable polymerases for the present invention may have varying levels of fidelity. In some embodiments, polymerases in accordance with the present invention have high-fidelity. Suitable polymerases for the present invention may have varying levels of processivity. In some embodiments, polymerases in accordance with the present invention have high processivity.

Typically, a suitable polymerase can carry out extensive DNA synthesis on both strands of a DNA template, with the synthesized DNA in turn being capable of being used as a template for new DNA synthesis. This results in an exponential increase in the amount of DNA synthesized with time. Strand-displacement activity is important for the formation of branched amplification on double-stranded nucleic acids, which typically lead to exponential amplification of template nucleic acid. Suitable polymerases for the present invention may however have varying levels of strand-displacement activity. In some embodiments, suitable polymerases for the present invention have high strand-displacement activity. One non-limiting example of polymerases with high strand-displacement activity is Bacillus bacteriophage Phi29 DNA polymerase. Phi29 DNA polymerase is very processive and generates DNA up to 70 kb in length using M13 DNA as a template. In some embodiments, suitable polymerases exhibit low or no strand displacement activity. Such polymerases are particularly useful if they are thermophilic and/or thermostable. For example, DNA amplification can be carried out under thermocycling conditions using such polymerases in combination with heat denaturing. Examples of polymerases that are hyperthermophilic and/or thermostable but with low or no strand displacement activity include, but are not limited to Taq polymerase, Tth polymerase, Kapa2G polymerase (Kapa Biosystems).

In some embodiments, polymerases suitable for the present invention are thermostable, have high-fidelity and exhibits high strand-displacement activity. Non-limiting examples of polymerases with these characteristics are the wild-type and exonuclease minus version of Pyrophage 3173 (US Patent publication 20080268498 by Lucigen, the disclosure of which is incorporated by reference in its entirety). Other examples include, but are not limited to, KOD polymerase (Novagen), Vent and DeepVent polymerases (New England Biolabs) and KapaHiFi (Kapa Biosystems).

In some embodiments, a moderately thermostable polymerase can be used. A non-limiting example of such polymerase is Bst polymerase. Typically, such moderate thermostable polymerase can be used in conjunction with low-temperature melting reagents so that DNA can be denatured at a lower temperature compatible with a less thermostable polymerase and/or primase. Suitable low-temperature melting reagents include, but are not limited to, betaine, DMSO and glycerol. Additionally or alternatively, a thermoprotectant can be used in conjunction with a less thermostable polymerase to stabilize the enzyme at higher temperature. Suitable thermoprotectants include, but are not limited to, ectoine, hydroxy ectoine, mannosylglycerate, trehalose, betaine, glycerol and proline.

Additional polymerases suitable for the present invention include both type A and type B DNA polymerases. Examples of type B polymerases suitable for the invention include, but are not limited to, DNA polymerases from archaea (e.g., Thermococcus litoralis (Vent™, GenBank: AAA72101), Pyrococcus furiosus (Pfu, GenBank: D12983, BAA02362), Pyrococcus woesii, Pyrococcus GB-D (Deep Vent™, GenBank: AAA67131), Thermococcus kodakaraensis KODI (KOD, GenBank: BD175553; Thermococcus sp. strain KOD (Pfx, GenBank: AAE68738, BAA06142)), Thermococcus gorgonarius (Tgo, Pdb: 4699806), Sulfolobus solataricus (GenBank: NC002754, P26811), Aeropyrum pernix (GenBank: BAA81109), Archaeglobus fulgidus (GenBank: 029753), Pyrobaculum aerophilum (GenBank: AAL63952), Pyrodictium occultum (GenBank: BAA07579, BAA07580), Thermococcus 9 degree Nm (GenBank: AAA88769, Q56366), Thermococcus fumicolans (GenBank: CAA93738, P74918), Thermococcus hydrothermalis (GenBank: CAC18555), Thermococcus spp. GE8 (GenBank: CAC12850), Thermococcus spp. JDF-3 (GenBank: AX135456; WO0132887), Thermococcus spp. TY (GenBank: CAA73475), Pyrococcus abyssi (GenBank: P77916), Pyrococcus glycovorans (GenBank: CAC12849), Pyrococcus horikoshii (GenBank: NP 143776), Pyrococcus spp. GE23 (GenBank: CAA90887), Pyrococcus spp. ST700 (GenBank: CAC 12847), Thermococcus pacificus (GenBank: AX411312.1), Thermococcus zilligii (GenBank: DQ3366890), Thermococcus aggregans, Thermococcus barossii, Thermococcus celer (GenBank: DD259850.1), Thermococcus profundus (GenBank: E14137), Thermococcus siculi (GenBank: DD259857.1), Thermococcus thioreducens, Thermococcus onnurineus NA1, Sulfolobus acidocaldarium, Sulfolobus tokodaii, Pyrobaculum calidifontis, Pyrobaculum islandicum (GenBank: AAF27815), Methanococcus jannaschii (GenBank: Q58295), Desulforococcus species TOK, Desulfurococcus, Pyrolobus, Pyrodictium, Staphylothermus, Vulcanisaetta, Methanococcus (GenBank: P52025) and other archaeal B polymerases, such as GenBank AAC62712, P956901, BAAA07579)). Additional representative temperature-stable family A and B polymerases include, e.g., polymerases extracted from the thermophilic bacteria Thermus species (e.g., flavus, ruber, thermophilus, lacteus, rubens, aquaticus), Bacillus stearothermophilus, Thermotoga maritima, Methanothermus fervidus.

In some embodiments, DNA polymerases suitable for the invention are type A DNA polymerases. Examples of suitable type A polymerases include, but are not limited to, E. coli pol I (e.g., Klenow fragment), Thermus aquaticus DNA pol I (Taq polymerase), Thermus flavus DNA pol I, Streptococcus pneumoniae DNA pol I, Bacillus stearothermophilus pol I, phage polymerase T5, phage polymerase T7, mitochondrial DNA polymerase pol gamma, as well as polymerases obtained from the following: Geobacillus stearothermophilus (ACCESSION 3BDP_A; VERSION 3BDP_A; GI:4389065; DBSOURCE pdb: molecule 3BDP, chain 65, release Aug. 27, 2007), Natranaerobius thermophilus JW/NM-WN-LF (ACCESSION ACB8546; VERSION ACB85463.1; GI:179351193; DBSOURCE accession CP001034.1), Thermus thermophilus HB8 (ACCESSION P52028; VERSION P52028.2; GI:62298349; DBSOURCE swissprot: locus DPO1T_THET8, accession P52028), Thermus thermophilus (ACCESSION P30313; VERSION P30313.1; GI:232010; DBSOURCE swissprot: locus DPO1F_THETH, accession P30313), Thermus caldophilus (ACCESSION P80194; VERSION P80194.2; GI:2506365; DBSOURCE swissprot: locus DPO1_THECA, accession P80194), Thermus filiformis (ACCESSION 052225; VERSION 052225.1; GI:3913510; DBSOURCE swissprot: locus DPO1_THEFI, accession 052225), Thermus filiformis (ACCESSION AAR11876; VERSION AAR11876.1; GI:38146983; DBSOURCE accession AY247645.1), Thermus aquaticus (ACCESSION P19821; VERSION P19821.1; GI:118828; DBSOURCE swissprot: locus DPO1_THEAQ, accession P19821), Thermotoga lettingae TMO (ACCESSION YP_{—}001469790; VERSION YP_{—}001469790.1; GI:157363023; DBSOURCE REFSEQ: accession NC_{—}009828.1), Thermosipho melanesiensis B1429 (ACCESSION YP_{—}001307134; VERSION YP_{—}001307134.1; GI:150021780; DBSOURCE REFSEQ: accession NC_{—}009616.1), Thermotoga petrophila RKU-1 (ACCESSION YP_{—}001244762; VERSION YP_{—}001244762.1; GI:148270302; DBSOURCE REFSEQ: accession NC_{—}009486.1), Thermotoga maritima MSB8 (ACCESSION NP_{—}229419; VERSION NP_{—}229419.1; GI:15644367; DBSOURCE REFSEQ: accession NC_{—}000853.1), Thermodesulfovibrio yellowstonii DSM 11347 (ACCESSION YP_{—}002249284; VERSION YP_{—}002249284.1; GI:206889818; DBSOURCE REFSEQ: accession NC_{—}011296.1), Dictyoglomus thermophilum (ACCESSION AAR11877; VERSION AAR11877.1; GI:38146985; DBSOURCE accession AY247646.1), Geobacillus sp. MKK-2005 (ACCESSION ABB72056; VERSION ABB72056.1; GI:82395938; DBSOURCE accession DQ244056.1); Bacillus caldotenax (ACCESSION BAA02361; VERSION BAA02361.1; GI:912445; DBSOURCE locus BACPOLYTG accession D12982.1); Thermoanaerobacter thermohydrosulfuricus (ACCESSION AAC85580; VERSION AAC85580.1; GI:3992153; DBSOURCE locus AR003995 accession AAC85580.1), Thermoanaerobacter pseudethanolicus ATCC 33223 (ACCESSION ABY95124; VERSION ABY95124.1; GI:166856716; DBSOURCE accession CP000924.1), Enterobacteria phage T5 (ACCESSION AAS77168 CAA04580; VERSION AAS77168.1; GI:45775036; DBSOURCE accession AY543070.1) and Enterobacteria phage T7 (T7) (ACCESSION NP_{—}041982; VERSION NP_{—}041982.1; GI:9627454; DBSOURCE REFSEQ: accession NC_{—}001604.1).

In some embodiments, DNA polymerases suitable for the present invention are chimeric polymerases, fusion polymerases or other modified polymerases, such as, for example, those described in PCT/US09/63166, PCT/US09/63167, and PCT/US09/63169, the contents of each of which are incorporated herein by reference.

The sequences of the polymerases described herein are readily accessible through public databases using the accession no. described herein. All the sequences are incorporated herein by reference in their entireties. Exemplary sequences are provided in the Examples section. Suitable polymerases for the invention also include various functional variants of the polymerases described herein including variants having an amino acid sequence at least 70%, 75%, 80%, 85%, 90%, 95%, 96%, 97%, 98%, 99% identical to corresponding sequence provided herein.

In some embodiments, two or more polymerases described herein can be used in an amplification reaction according to the invention. For example, polymerases with different characteristics (e.g., high strand-displacement activity, high fidelity, high processivity, or high thermostability) can be combined to optimize amplification results.

Accessory proteins

Although not required, accessory proteins can be included in amplification reactions according to the invention. Typically, accessory proteins include, but are not limited to, processivity factors, helicases, and DNA binding proteins such as ssDNA binding proteins (for review, see Kornberg and Baker, DNA Replication, Freeman and Co., New York, 1992). In some embodiments, addition of accessory proteins will result in efficient DNA synthesis.

DNA Helicase

Helicase may help unwind DNA template and/or strand displacement. In some embodiments, helicase may replace heat denaturing to separate double-stranded DNA. Typically, helicase interacts specifically with DNA polymerase during amplification. The energy for helicase activity is typically obtained by the hydrolysis of nucleoside triphosphates.

Suitable helicases can be derived from a prokaryote or a eukaryote. For example, the DNA helicase can be from a bacterium such as E. coli, a bacteriophage such as bacteriophage T4 or bacteriophage T7, a yeast, or human. Exemplary helicases include, but are not limited to, the bacteriophage T4 gene product 41, the bacteriophage T4 dda protein, the bacteriophage T7 gene 4 protein, the E. coli UvrD protein, the E. coli dnaB protein, and any mutants or functional variants thereof, including those described in Salinas and Kodadek, 1995 Cell 82(1):111-9; Salinas and Benkovic, 2000 Proc Natl Acad Sci USA; 97(13):7196-201; Alberts, et al., 1983 Cold Spring Harb Symp Quant Biol. 47 Pt 2:655-68, all of which are herein incorporated by reference.

One example of helicases suitable for the present invention is bacteriophage T7 the gene 4 protein. Its preferred substrate for hydrolysis is dTTP. The phage makes two forms of the gene 4 protein of molecular weight 56,000 and 63,000; the two forms arise from two in-frame start codons. As discussed above, the 63-kDa form of the gene 4 protein also provides primase activity (Bernstein and Richardson, 1989 J. Biol. Chem. 264:13066). Modified forms containing substitutions, insertions, deletions, in the 63-kDa protein are also suitable for the present invention. One non-limiting example of an altered helicase enzyme is the 63-kDa gene 4 protein in which the methionine at residue 64 is changed to a glycine (g4_{G64}). (Mendelman et al., 1992 Proc. Natl. Acad. Sci. USA 89:10638; Mendelman et al., 1993 J. Biol. Chem. 268:27208). All enzymatic properties of the g4_{G64} form of the gene 4 protein that have been examined are comparable to those of the wild-type 63-kDa gene 4 protein, including its use as a primase and helicase for amplification as described in the current invention.

In some embodiments, an ATP-regeneration system may be added to amplification reactions when a helicase is used. During some DNA synthesis reactions, some of the deoxynucleoside triphosphates will be degraded to deoxynucleoside diphosphates due to hydrolysis by the helicase, if present. The degradation of deoxynucleoside triphosphates can be minimized by the use of an ATP regeneration system which, in the presence of nucleoside diphosphokinase, will convert any nucleoside diphosphate in the reaction mixture to the triphosphate. For example, in the T7 replication system, the helicase very rapidly degrades dTTP to dTDP for energy. The presence of an ATP-regeneration system will increase the amount of nucleotides capable of serving as precursors for DNA synthesis.

A number of ATP regeneration systems suitable for the invention are known in the art. For example, the combination of phosphocreatine (Sigma Chemical Co., St. Louis, Mo.) and creatine kinase (Sigma Chemical Co., St. Louis, Mo.) will push the equilibrium between ADP and ATP towards ATP, at the expense of the phosphocreatine.

Single-Stranded DNA Binding Protein

Single-stranded DNA (ssDNA) binding (SSB) proteins may serve a number of roles, including, for example, removal of secondary structure from single-stranded DNA to allow efficient DNA synthesis and prevent pre-mature annealing (for review, see Kornberg and Baker, DNA Replication, Freeman and Co., New York, 1992). Suitable SSB proteins can be isolated from various organisms from viruses to humans. Exemplary SSB proteins suitable for the invention include, but are not limited to, SSB protein from E. coli, gene 2.5 protein from bacteriophage T7 (Kim et al., 1992 J. Biol. Chem. 267:15022), RPA (Replication Protein A) from eukaryotes, SSB from Sulfolobus Solfataricus and phage T4 gene 32 protein.

Typically, SSB proteins can improve the processivity of DNA polymerase, for example, during isothermal amplification, particularly at temperatures below 30° C. (Tabor et al., 1987 J. Biol. Chem. 262:16212). In some embodiments, the amount of SSB protein for a 50 μl reaction is from 0.01 to 1 μg. In some embodiments, the presence of SSB proteins stimulates the rate of DNA synthesis by several fold (e.g., more than 2-fold, 3-fold, 4-fold, 5-fold, or 6-fold).

Nucleoside Diphosphokinase

In general, nucleoside diphosphokinase rapidly transfers the terminal phosphate from a nucleoside triphosphate to a nucleoside diphosphate. Nucleoside diphosphokinase is relatively nonspecific for the nucleoside, recognizing all four ribo- and deoxyribonucleosides. Thus it efficiently equilibrates the ratio of nucleoside diphosphates and nucleoside triphosphates among all the nucleotides in the mixture. It is thought that this enzyme can increase the amount of DNA synthesis if one of the required nucleoside triphosphates is preferentially hydrolyzed during the reaction. Exemplary nucleoside diphosphokinases suitable for the invention include, but are not limited to, nucleoside diphosphokinase from Baker's Yeast (Sigma Chemical Co., St. Louis, Mo.), nucleoside diphosphokinase purified from E. coli (described by Almaula, et al. 1995 J. Bact. 177:2524). Other nucleoside diphosphokinases are known to those who practice the art and can be used in the present invention.

Inorganic Pyrophosphatase

In some DNA amplification reactions, inorganic pyrophosphate will accumulate as a product of the reactions. If the concentration becomes too high, it can reduce the amount of DNA synthesis due to product inhibition. The accumulation of inorganic pyrophosphate can be prevented by the addition of inorganic pyrophosphatase. Exemplary inorganic pyrophosphatase suitable for the present invention include yeast inorganic pyrophosphatase (Sigma Chemical Co., St. Louis, Mo.). Other inorganic pyrophosphatases are known in the art and can be used in the present invention.

### Amplification Conditions

In some embodiments, amplification reactions according to the present invention are carried out under substantially constant temperature, i.e., isothermal conditions. Isothermal amplification relies on methods other than thermocycling to denature the DNA, such as the strand displacement activity of some polymerases or DNA helicases. Thus, isothermal amplification does not mean that no temperature fluctuation occurs during amplification, but rather indicates that the temperature variation during the amplification process is not sufficiently great to provide the predominant mechanism to denature product/template hybrids.

Suitable temperature for an isothermal amplification reaction can be determined according to several factors, including, for example, the optimal temperature for enzymatic activity and template nucleotide composition, for example, GC composition. In some embodiments, a suitable temperature for isothermal amplification is at or less than 60° C. (e.g., at or less than 50° C., 45° C., 40° C., 37° C., 35° C., 30° C., 25° C., 20° C.).

In some embodiments, isothermal amplification is preceded by a pre-incubation step at a different temperature. For example, in some embodiments, nucleic acid amplification mixture (e.g., with or without polymerase added) is pre-incubated at a lower temperature (e.g., 20, 21, 22, 23, 24, 25, 26, 27, 28, 29, 30, or more ° C.) for a given time (e.g., 5, 10, 15, 20, 25, 30, 45, 60 or more minutes) before being brought to a higher temperature for amplification (e.g., 30, 35, 30, 45, 50 or more ° C.). In some embodiments, nucleic acid amplification mixture (e.g., with or without polymerase added) is pre-incubated at a higher temperature (e.g., 65, 70, 75, 80, 85, 90, 95, or more ° C.) for a given time (e.g., 5, 10, 15, 20, 25, 30, or more minutes) before being brought to a lower temperature for amplification (e.g., 30, 35, 30, 45, 50 or more ° C.).

In some embodiments, nucleic acid amplification reactions according to the present invention are carried out under thermocycling conditions similar to those conditions for PCR amplification. In some embodiments, thermocycling conditions contain a series of 20 to 40 repeated temperature cycles. Each thermocycle typically includes 2-3 discrete temperature steps including at least heat denaturing step at a higher temperature (e.g., at or above 90 or 95° C.) and primer and/or DNA synthesis at lower temperatures (e.g., 50° C. for primer synthesis and 72° C. for DNA synthesis). A typical cycle includes 15 minutes at 72° C., 30 seconds at 95° C., 1 minute at 50° C. The temperature ranges of thermocycling can vary according to factors, such as, template DNA composition, concentration of divalent ions and dNTPs, additional components added to the reaction mixture, optimal temperature for primase and polymerase activity, etc.

### Whole Genome Amplification

The present invention may be utilized to amplify any nucleic acid. The present invention is particularly useful for whole genome amplification (also known as global nucleic acid amplification).

The invention provides methods for whole genome amplification that can be used to amplify genomic DNA prior to genetic evaluation such as detection of typable loci in the genome. Whole genome amplification methods of the invention can be used to increase the quantity of genomic DNA without compromising the quality or the representation of any given sequence. Thus, the methods can be used to amplify a relatively small quantity (e.g., trace amount) of genomic DNA to provide levels of the genomic DNA that can be genotyped or further analyzed. In some embodiments, the present invention can be used to amplify nucleic acids in a sample at a concentration at or less than, for example, 300 ng/μl, 200 ng/μl, 150 ng/μl, 100 ng/μl, 95 ng/μl, 90 ng/μl, 85 ng/μl, 80 ng/μl, 75 ng/μl, 70 ng/μl, 65 ng/μl, 60 ng/μl, 55 ng/μl, 50 ng/μl, 45 ng/μl, 40 ng/μl, 35 ng/μl, 30 ng/μl, 25 ng/μl, 20 ng/μl, 15 ng/μl, 10 ng/μl, 5 ng/μl, 1 ng/μl, 0.5 ng/μl, or 0.1 ng/μl. In some embodiments, the present invention can be used to amplify nucleic acids in a sample in an amount of or less than, for example, 500 ng, 450 ng, 400 ng, 350 ng, 300 ng, 250 ng, 200 ng, 150 ng, 100 ng, 50 ng, 10 ng, or 1 ng. In some embodiments, the present invention can be used to amplify a genome in a sample, and the genome can constitute any fraction of the total nucleic acids in the sample. For example, the genome can constitute, for example, less than 100%, 95%, 90%, 85%, 80%, 75%, 70%, 65%, 60%, 55%, 50%, 45%, 40%, 35%, 30%, 25%, 20%, 15%, 10%, 5%, 1%, or 0.1% of the total nucleic acids in the sample.

In some embodiments, the present invention provides amplification of genomic DNA such that the amount of amplified product is at least about 10-fold greater, or at least 100-fold greater, or at least 1000-fold greater, or at least 10,000-fold greater, or at least 100,000-fold greater, or at least 1,000,000-fold greater, or at least 10,000,000-fold greater or even more than the amount of DNA in the original sample.

In some embodiments, the present invention can be used to amplify a complex genome. In particular, the present invention can accurately and evenly amplify various sequences in highly complex nucleic acid samples. The quality of the amplification products can also be measured in a variety of ways, including, but not limited to, genomic coverage, amplification bias, allele bias, locus representation, sequence representation, allele representation, locus representation bias, sequence representation bias, percent representation, percent locus representation, percent sequence representation, and other measure that indicate unbiased and/or complete amplification of the input nucleic acids.

Genome coverage generally refers to the percent of template nucleotide (i.e., genome) that is amplified in a given amplification reaction. Methods for determining genome coverage are known in the art (see, for example, Pinard, et al., 2006 BMC Genomics 7:216, the entire contents of which is herein incorporated by reference). In some embodiments, inventive methods according to the present invention result in genome coverage that is greater than 15%, 20%, 25%, 30%, 35%, 40%, 45%, 50%, 55%, 60%, 65%, 70%, 75%, 80%, 85%, 90%, 95%, 98% or more.

In some embodiments, the efficiency of a DNA amplification procedure may be described for individual loci as the percent representation. The percent representation is 100% for a locus in genomic DNA when the genomic DNA was purified from cells. Amplification bias may be calculated between two samples of amplified DNA or between a sample of amplified DNA and the template DNA from which it was amplified. The bias is the ratio between the values for percent representation (or for locus representation) for a particular locus. The maximum bias is the ratio of the most highly represented locus to the least represented locus. Other methods for determination of amplification bias are known in the art. See, for example, Pinard, et al., 2006 BMC Genomics 7:216, which is incorporated herein by reference.

Inventive methods according to the present invention can produce high quality amplification products. For example, inventive methods of the invention can produce amplified genome product with a locus representation of at least 15%, at least 20%, at least 25%, at least 30%, at least 35%, at least 40%, at least 45%, at least 50%, at least 60%, at least 70%, at least 80%, at least 90%, or at least 100% for at least 5 different loci. In some embodiments, inventive methods of the invention can produce amplified genome product with a locus representation of at least 10% for at least 6 different loci, at least 10 different loci, at least 15 different loci, at least 20 different loci, at least 25 different loci, at least 30 different loci, at least 40 different loci, at least 50 different loci, at least 75 different loci, or at least 100 different loci.

In some embodiments, inventive methods of the invention can produce amplified genome product with sequence representation of at least 15%, at least 20%, at least 25%, at least 30%, at least 35%, at least 40%, at least 45%, at least 50%, at least 60%, at least 70%, at least 80%, at least 90%, or at least 100% for at least 5 different target sequences. In some embodiments, inventive methods of the invention can produce amplified genome product with sequence representation of at least 10% for at least 6 different target sequences, at least 10 different target sequences, at least 15 different target sequences, at least 20 different target sequences, at least 25 different target sequences, at least 30 different target sequences, at least 40 different target sequences, at least 50 different target sequences, at least 75 different target sequences, or at least 100 different target sequences.

In some embodiments, inventive methods of the present invention can produce amplified genome product with an amplification bias of less than 45-fold, less than 40-fold, less than 35-fold, less than 30-fold, less than 25-fold, less than 20-fold, less than 15-fold, less than 10-fold, less than 5-fold for at least 5 different loci or target sequences. In some embodiments, inventive methods of the present invention can produce amplified genome product with an amplification bias of less than 50-fold for at least 5 different loci or target sequences, at least 10 different loci or target sequences, at least 15 different loci or target sequences, at least 20 different loci or target sequences, at least 25 different loci or target sequences, at least 30 different loci or target sequences, at least 40 different loci or target sequences, at least 50 different loci or target sequences, at least 75 different loci or target sequences, or at least 100 different loci or target sequences.

The length of amplified DNA is also an important factor for downstream applications. In some embodiments, inventive methods of the present invention provide amplified genomic fragments that are at least about 5 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb or more in length.

In some embodiments, the amplification products are labeled to facilitate detection. Exemplary properties of suitable labels upon which detection can be based include, but are not limited to, mass, electrical conductivity, energy absorbance, fluorescence or the like. In some embodiments, one or more detectably labeled nucleotides can be added to amplification reactions so that they can be incorporated into amplification products. Non-limiting examples of label moieties useful for the invention include, without limitation, fluorophores such as umbelliferone, fluorescein, fluorescein isothiocyanate, rhodamine, tetramethyl rhodamine, eosin, green fluorescent protein, erythrosin, coumarin, methyl coumarin, pyrene, malachite green, stilbene, lucifer yellow, Cascade Blue™, Texas Red, dichlorotriazinylamine fluorescein, dansyl chloride, phycoerythrin, fluorescent lanthanide complexes such as those including Europium and Terbium, Cy3, Cy5, SYBR Green II, molecular beacons and fluorescent derivatives thereof, as well as others known in the art as described, for example, in Principles of Fluorescence Spectroscopy, Joseph R. Lakowicz (Editor), Plenum Pub Corp, 2nd edition (July 1999) and the 6th Edition of the Molecular Probes Handbook by Richard P. Hoagland; a luminescent material such as luminol; light scattering or plasmon resonant materials such as gold or silver particles or quantum dots; or radioactive material include ¹⁴C_{,} ¹²³I, ¹²⁴I, ¹²⁵I, ¹³¹I, Tc99m, ³⁵S or ³H; or suitable enzymes such as horseradish peroxidase, alkaline phosphatase.

The products from whole genome amplification according to the present invention can be used for various down stream analysis including, but not limited to, analysis of nucleic acids present in cells (for example, analysis of genomic DNA in cells) and on genomic DNA arrays, disease detection including prenatal diagnosis (for example, detection of inherited diseases such as cystic fibrosis, muscular dystrophy, diabetes, hemophilia, sickle cell anemia; assessment of predisposition for cancers such as prostate cancer, breast cancer, lung cancer, colon cancer, ovarian cancer, testicular cancer, pancreatic cancer), mutation detection, gene discovery, sequencing, gene mapping (molecular haplotyping), and copy-number-variation analysis (CNV).

### Kits

The invention also contemplates kit formats which include a package unit having one or more containers containing a primase and a polymerase described herein. In some embodiments, inventive kits of the invention further include various accessory proteins such as helicase, ssDNA-binding proteins, nucleoside diphosphokinase, reagents involved in ATP regeneration system, and/or other reagents useful for nucleic acid synthesis such as nucleotides (e.g., dNTPs), buffers, among others. Inventive kits in accordance with the present invention may also contain instructions and controls. Kits may include containers of reagents mixed together in suitable proportions for performing the methods in accordance with the invention. Reagent containers preferably contain reagents in unit quantities that obviate measuring steps when performing the subject methods.

### EXAMPLES

### Example 1

### Cloning of Tpol Polymerase and ORF904 Primase Fragment

An exemplary polymerase suitable for use in the present invention is pol-11 (Tpol) isolated from a Thermus species by Hjorleifsdottir, et al. (U.S. patent application Ser. No. 11/662,879, the disclosure of which is hereby incorporated by reference). This enzyme is moderately thermostable, has a very high specific activity, 3′ exonuclease activity, and strand-displacement activity. This enzyme has been used for WGA using random primers (U.S. patent application Ser. No. 11/662,879). A codon-optimized gene for Tpol (SEQ ID NO: 1) was synthesized by GeneArt and cloned into our expression vector pKB. The amino acid sequence of the coding region of the expression construct is given in SEQ ID NO: 2.

| Nucleotide Sequence of Tpol (SEQ ID NO: 1): | |
|---|---|
| 1 | ATGGCTAGCG CCGAAGGTTT TGAACTGCAT TATATTCCGG AAGTTGGTCC GGGTATGGGT |
| 61 | GAACTGCTGG ATCTGCTGAT GCGTCAGCCG GTTCTGGGTG TTGATCTGGA AACCACCGGT |
| 121 | CTGGATCCGC ATACCAGCCG TCCGCGTCTG CTGTCTCTGG CCATGCCTGG TGCAGTTGTT |
| 181 | GTTTTTGACC TGTTTGGTGT TCCGCTGGAA GTTTTTTATC CGCTGTTTAG CCGTGAAGAA |
| 241 | GGTCCGCTGC TGGTTGGTCA TAATCTGAAA TTTGATCTGC TGTTTCTGCT GAAAGCAGGT |
| 301 | GTTTGGCGTG CAAGCGGTAA ACGTCTGTGG GATACCGGTC TGGCCCATCA GGTTCTGCAT |
| 361 | GCACAGGCAC GTATGCCTGC ACTGAAAGAT CTGGCTCCGG GTCTGGATAA AACCCTGCAG |
| 421 | ACCAGCGATT GGGGTGGTCC GCTGTCTAGC GAACAGGTTG CATATGCAGC ACTGGATGCA |
| 481 | GCAGTTCCGC TGGTTCTGTA TCGTGAACAG CGTGAACGTG CACGTACCCT GCGTCTGGAA |
| 541 | AAAGTTCTGG AAGTTGAACG TCGTGCACTG CCTGCAGTTG CATGGATGGA ACTGCGTGGT |
| 601 | GTTCCGTTTG CACCGGAACT GTGGGAAGAA GCAGCACGCG AAGCAGAACG TGAAGCCGAA |
| 661 | GCACTGCGTG GTGAACTGCC GTTTGGTGTT AATTGGAATT CTCCGGCACA GGTTCTGGCC |
| 721 | TATCTGAAAG GTGAAGGTCT GGATCTGCCG GATACCCGTG AAGATACCCT GGCTGGTTAT |
| 781 | CGTGAACATC CGCTGGTTGC AAAACTGCTG CGTTATCGCG AAGCAGCAAA ACGTGTTAGC |
| 841 | ACCTATGGTA AAGAATGGGC CAAACATCTG AATCCGGCAA CCGGTCGTAT TCATCCGAGC |
| 901 | TGGCAGCAGA TTGGTGCAGA AACCGGTCGC ATGGCATGTC GTAAACCGAA TCTGCAGCAG |
| 961 | GTTCCGCGTG ATCCGGCACT GCGTCGTGCA TTTCGTCCGA AAGAAGGTCG TGTTATGCTG |
| 1021 | AAAGCCGATT TTAGCCAGAT TGAACTGCGT ATTGCAGCAG CAATTGCAAA AGAAGGTCGC |
| 1081 | ATGCTGCGCG CCTTTCGTGA AGGTAAAGAT CTGCATGCAC TGACCGCAAG CCTGGTTCTG |
| 1141 | GGTAAACCGC TGGAAGAAGT GGGTAAAGAA GATCGTCAGC TGGCCAAAGC ACTGAATTTT |
| 1201 | GGTCTGCTGT ATGGTCTGGG TGCAGAAGGT CTGCGTCGTT ACGCCCTGAC CGCATATGGT |
| 1261 | GTTAAACTGA CCCTGGAAGA AGCACAGAAA CTGCGCGATG CATTTTTTCG TGCATATCCG |
| 1321 | GCTCTGAAAC GTTGGCATCG TAGCCAGCCG GAAGGTGAAG TTGTTGTTCG TACCCTGCTG |
| 1381 | GGTCGTCGTC GTACCACCGA TCGTTATACC GAAAAACTGA ATACACCGGT TCAGGGCACC |
| 1441 | GGTGCAGATG GTCTGAAAAT GGCACTGGCC CTGCTGTGGG AAAATCGTGG TCTGCTGTGG |
| 1501 | GGTGCATTTC CGGTTCTGGC CGTTCATGAT GAAGTTGTTC TGGAAGCACC GGAAGAAGGT |
| 1561 | GCAAAAGAAT ATCTGGAAAC CCTGACCGCA CTGATGCGCC AGGGTATGGA AGAAGTTCTG |
| 1621 | GGCGGCGCAG TTCCGGTTGA AGTTGAAGGT GGTATTTATC GTGATTGGGG TGCAACACCG |
| 1681 | TGGGAAGAGG CCTAA |
| Amino Acid Sequence of Tpol (SEQ ID NO: 2): | |
| 1 | MASAEGFELH YIPEVGPGMG ELLDLLMRQP VLGVDLETTG LDPHTSRPRL LSLAMPGAVV |
| 61 | VFDLFGVPLE VFYPLFSREE GPLLVGHNLK FDLLFLLKAG VWRASGKRLW DTGLAHQVLH |
| 121 | AQARMPALKD LAPGLDKTLQ TSDWGGPLSS EQVAYAALDA AVPLVLYREQ RERARTLRLE |
| 181 | KVLEVERRAL PAVAWMELRG VPFAPELWEE AAREAEREAE ALRGELPFGV NWNSPAQVLA |
| 241 | YLKGEGLDLP DTREDTLAGY REHPLVAKLL RYREAAKRVS TYGKEWAKHL NPATGRIHPS |
| 301 | WQQIGAETGR MACRKPNLQQ VPRDPALRRA FRPKEGRVML KADFSQIELR IAAAIAKEGR |
| 361 | MLRAFREGKD LHALTASLVL GKPLEEVGKE DRQLAKALNF GLLYGLGAEG LRRYALTAYG |
| 421 | VKLTLEEAQK LRDAFFRAYP ALKRWHRSQP EGEVVVRTLL GRRRTTDRYT EKLNTPVQGT |
| 481 | GADGLKMALA LLWENRGLLW GAFPVLAVHD EVVLEAPEEG AKEYLETLTA LMRQGMEEVL |
| 541 | GGAVPVEVEG GIYRDWGATP WEEA |

An exemplary primase suitable for use in the present invention is the ORF904 primase as described by Lipps and co-workers (Lipps, et al. 2003, EMBO, 22(10): 2516-2525). This thermostable primase was identified on a plasmid from Sulfolobus islandicus. The primase initiates primer synthesis at a tri-nucleotide GTG recognition motif. It utilizes primarily dNTPs for primer synthesis and it is thought that it requires at least one ribonucleotide for primer synthesis. Synthesized primers are typically around 8 nucleotides long and can be further extended by the primase or heterogeneously added DNA polymerases (e.g., Taq DNA polymerase). The full Open Reading Frame (ORF) of the primase encodes a protein with 904 amino acids in which part of the N-terminal domain has homology to primases and polymerases and the C-terminal domain has homology to helicases. A truncation encompassing amino acid residues 1 to 370 has primase activity and does not include the region with homology to helicases (Beck et al. 2007, Nucleic Acid Research 17:5635-5645).

The N-terminal 370 amino acids of the ORF904 primase were codon-optimized and the gene was synthesized by Mr Gene, Gmbh (Regensburg, Germany). The truncated ORF904 was cloned into a vector for expression in E. coli (SEQ ID NO: 3 and SEQ ID NO: 4). The gene was expressed in E. coli and the primase was purified using exemplary purification method given by Beck et al. (Beck et al., 2007, Nucleic Acid Research 17:5635-5645). The concentration and purity of the ORF904 primase and of Tpol polymerase was determined on a 2100 BioAnalyzer chip (Agilent Technologies).

| Nucleotide Sequence of Truncated ORF904 Primase (SEQ ID NO: 3): | |
|---|---|
| 1 | ATGGCTAGCG CCATTAATAA ACGCAGCAAA GTGATTCTGC ATGGCAATGT GAAAAAAACC |
| 61 | CGTCGTACCG GTGTTTATAT GATTAGCCTG GATAATAGCG GCAATAAAGA TTTTAGCAGC |
| 121 | AATTTTAGCA GCGAACGTAT TCGCTATGCA AAATGGTTTC TGGAACATGG CTTTAATATT |
| 181 | ATTCCGATTG ATCCGGAAAG CAAAAAACCG GTTCTGAAAG AATGGCAGAA ATATAGCCAT |
| 241 | GAAATGCCGT CCGATGAAGA AAAACAGCGC TTTCTGAAAA TGATTGAAGA AGGCTATAAT |
| 301 | TACGCAATTC CGGGTGGTCA GAAAGGTCTG GTGATTCTGG ATTTTGAAAG CAAAGAAAAA |
| 361 | CTGAAAGCCT GGATTGGTGA AAGCGCACTG GAAGAACTGT GTCGTAAAAC CCTGTGTACC |
| 421 | AATACCGTTC ATGGTGGCAT TCATATTTAT GTTCTGAGCA ATGATATTCC GCCGCATAAA |
| 481 | ATTAATCCGC TGTTTGAAGA AAATGGCAAA GGCATTATTG ATCTGCAGAG CTATAATAGC |
| 541 | TATGTTCTGG GTCTGGGTAG CTGTGTTAAT CATCTGCATT GCACCACCGA TAAATGTCCG |
| 601 | TGGAAAGAAC AGAATTATAC CACCTGCTAT ACCCTGTATA ATGAACTGAA AGAAATTAGC |
| 661 | AAAGTGGATC TGAAAAGCCT GCTGCGTTTT CTGGCCGAAA AAGGTAAACG TCTGGGTATT |
| 721 | ACACTGAGCA AAACCGCAAA AGAATGGCTG GAAGGCAAAA AAGAAGAAGA AGATACCGTT |
| 781 | GTTGAATTTG AAGAACTGCG CAAAGAACTG GTTAAACGTG ATAGCGGTAA ACCGGTGGAA |
| 841 | AAAATTAAAG AAGAAATTTG CACCAAAAGC CCGCCGAAAC TGATTAAAGA AATTATTTGC |
| 901 | GAAAACAAAA CCTATGCCGA TGTGAATATT GATCGTAGCC GTGGTGATTG GCATGTTATT |
| 961 | CTGTATCTGA TGAAACATGG TGTTACCGAT CCGGATAAAA TTCTGGAACT GCTGCCGCGT |
| 1021 | GATAGCAAAG CAAAAGAAAA TGAAAAATGG AATACCCAGA AATATTTTGT GATTACCCTG |
| 1081 | AGCAAAGCAT GGTCTGTGGT GAAAAAATAT CTGGAAGCCT AA |
| Amino Acid Sequence of Truncated ORF904 Primase (SEQ ID NO 4): | |
| 1 | MASAINKRSK VILHGNVKKT RRTGVYMISL DNSGNKDFSS NFSSERIRYA KWFLEHGFNI |
| 61 | IPIDPESKKP VLKEWQKYSH EMPSDEEKQR FLKMIEEGYN YAIPGGQKGL VILDFESKEK |
| 121 | LKAWIGESAL EELCRKTLCT NTVHGGIHIY VLSNDIPPHK INPLFEENGK GIIDLQSYNS |
| 181 | YVLGLGSCVN HLHCTTDKCP WKEQNYTTCY TLYNELKEIS KVDLKSLLRF LAEKGKRLGI |
| 241 | TLSKTAKEWL EGKKEEEDTV VEFEELRKEL VKRDSGKPVE KIKEEICTKS PPKLIKEIIC |
| 301 | ENKTYADVNI DRSRGDWHVI LYLMKHGVTD PDKILELLPR DSKAKENEKW NTQKYFVITL |
| 361 | SKAWSVVKKY LEA* |

### Example 2

### DNA Amplification with ORF904 Primase and Taq or Tpol Polymerase

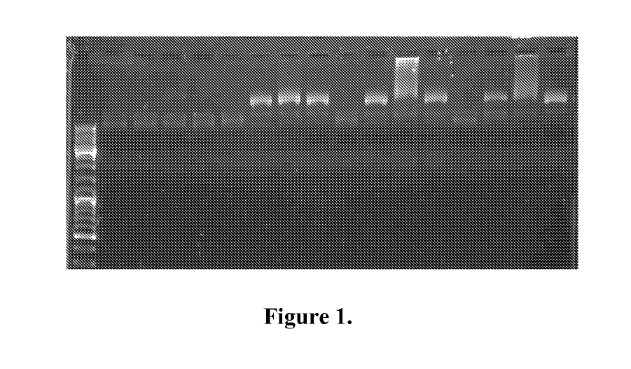

Whole-genome amplification was performed in 25 μl reactions containing: 20 mM Tris-HCl pH 8.8, 10 mM (NH₄)₂SO₄, 1.5 mM MgCl₂, 10 mM KCl, 2 mM MgSO₄, 0.1% Triton X-100, 0.2 mM dNTPs, 1 mM ATP and 180 ng M13mp18 ssDNA. 34 ng, 3.4 ng of ORF904 primase or 3 pmol of primer M13mp18-R (SEQ ID NO: 5) was added to the reactions together with 0.1 U of KapaTaq (KapaBiosystems) or 5 ng or 0.5 ng of Tpol. The samples were incubated for 60 minutes at 50° C. and 15 μl were run on an agarose gel (FIG. 1).

Oligo M13mp18-R (SEQ ID NO: 5):
AACGCCAGGGTTTTCCCAGTCACGACGTTGTAAAACGACG

The results show that in the absence of primer or primase there is little or no amplification by both Taq and Tpol polymerases. The bands similar in size to the largest band of the marker are template bands. The addition of primase or a primer results in a large increase in amplification seen as high molecular weight bands or smears. Tpol in particular yielded high molecular weight DNA. These results indicate that the primase produces primers that can be extended by either Taq or Tpol DNA polymerases.

### Example 3

### Phi29 Polymerase with ORF904 Primase

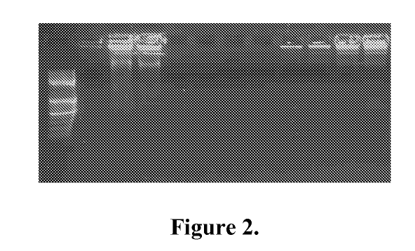

Phi29 DNA polymerase is characterized by high fidelity, processivity and strand-displacement activity. We used Phi29 polymerase together with ORF904 to amplify DNA without adding primers to the reactions. Whole-genome amplification was performed in 25 μl reactions containing 37 mM Tris-HCl, pH 8.0; 50 mM KCl, 10 mM MgCl₂, 5 mM (NH₄)₂SO₄, 1.0 mM dNTPs, 0.025 U yeast pyrophosphatase (Fermentas), 0.6× SYBR green (Roche), 1 mM ATP, 0.1 mM DTT and 15 ng M13 ssDNA. 100 ng Phi29 (Fermentas), ORF904 primase and/or random hexamers were added. The reactions were incubated at 30° C. in a RotorGene cycler (Corbett Life Science) for 200 cycles of 30 seconds with data acquisition after each cycle. The results show that amplification was achieved in the presence of Phi29 and random hexamers and with Phi29 and primase (FIG. 2). Very little amplification or no amplification was observed in the absence of primers, primase (lane 1) or polymerase (lanes 4-7). Adding increasing amounts of ORF904 primase gave increasing amounts of amplified DNA (lanes 8-11).

The specificity of amplification was confirmed through quantitative PCR (qPCR) with primers specific to M13mp18 phage DNA. 20 μl qPCR reactions using KapaSYBR Fast Universal were setup using 0.2 uM each of primers M13-20 (SEQ ID NO: 6) and M13 reverse (SEQ ID NO: 7). Two μl of a 1000-fold dilution of each WGA reaction was added to each qPCR reaction. A standard curve of 10-fold dilutions of M13 DNA between 20 ng/rxn and 20 fg/rxn was included. The qPCR reactions were incubated in a RotorGene thermocycler (Corbett Life Science) with the following cycling protocol: 3 min at 95° C., followed by 40 cycles of: (2 seconds at 95° C., 20 seconds at 60° C., data acquisition), and followed by meltcurve. The Phi29-only WGA reaction (lane 1) contained 8.6 pg in the qPCR. The no polymerase reactions (lanes 4-7) had 0.03-0.9 pg/reaction. The reactions with ORF904 and Phi29 had 21, 32, 88 or 113 pg M13 DNA/qPCR for WGA reactions 8-11 containing 50 ng, 150 ng, 500 ng and 1500 ng primase, respectively. Hence, ORF904 together with Phi29 increased the DNA amplification rate by 13-fold (to 113 pg/reaction) compared to the reaction with Phi29 only (8.6 pg/reaction).

M13-20 Primer (SEQ ID NO: 6):
GTAAAACGACGGCCAGT
M13 Reverse Primer (SEQ ID NO: 7):
GGAAACAGCTATGACCATG

### Example 4

### DNA Amplification with BstI Polymerase and ORF904 Primase

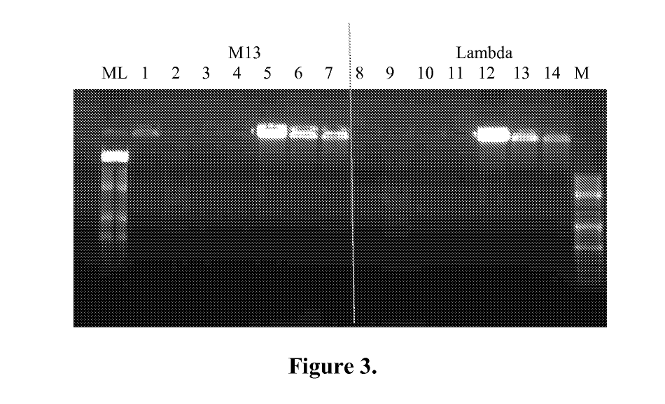

Whole-genome amplification was performed in 25 μl reactions containing 20 mM Tris-HCl pH 8.8, 10 mM (NH₄)₂SO₄, 10 mM KCl, 10 mM MgSO₄, 0.1% Triton X-100, 0.6× SYBR Green, 1 mM each dNTP, 50 uM ZnSO₄. The reactions each contained 5 ng of M13 ssDNA or lambda dsDNA template. In some reactions, the reaction mixtures contain ORF904 primase (500 ng, 750 ng or 1500 ng of ORF904 primase) and 8 U Bst polymerase (NEB) as indicated in the brief description of the drawings for FIG. 3. Some reactions contained 20 uM random hexamers. No-polymerase controls are also included. The reactions were incubated overnight at 50° C. and run on a 1% agarose gel.

The results are shown in FIG. 3. The gel shows that ORF904 primase stimulates DNA amplification in a dose-dependent manner in the presence of Bst DNA polymerase.

### Example 5

### DNA Amplification with Pyrophage 7130 Polymerase and ORF904 Primase

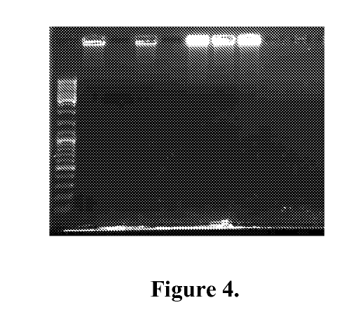

Whole-genome amplification was performed in 25 μl reactions containing 20 mM Tris-HCl pH 8.8, 10 mM (NH₄)₂SO₄, 10 mM KCl, 8 mM MgSO₄, 0.1% Triton X-100, 0.6× SYBR Green, 1 mM each dNTP, 50 uM ZnSO4, 1 uM DTT and 1.7 ng M13 ssDNA. Some reaction mixtures contained 20 uM random hexamers. Some reaction mixtures contained 50 ng or 500 ng ORF904 primase as indicated in the brief description of the drawings for FIG. 4. 5 U Bst polymerase and 0.1 mM NTPs were added to the reaction mixtures. No polymerase controls were also included. The reactions were incubated for 25 hours at 50° C. and the products were run on a 1% agarose gel. The results (FIG. 4) show that there is some DNA amplification in the absence of primers or primase but ORF904 primase greatly stimulates the amplification.

### Example 6

### Cloning of dnaG, E. coli Primase

dnaG is the primase involved in priming both leading and lagging strands during replication of the E. coli genome. It does not have helicase activity but interacts with a helicase, dnaB, during replication.

dnaG was PCR amplified from E. coli DH10B genomic DNA using primers DnaG-F (SEQ ID NO: 8) and DnaG-R (SEQ ID NO: 9). The primers contain Eco31I sites in their 5′ ends enabling directional cloning into our expression vector pKB. The construct was sequenced and the amino acid sequence of the coding region of dnaG is given as SEQ ID NO: 10. An example of expression and purification of dnaG is described by Khopde et al. (Biochemistry, 2002, 41, p 14820-14830).

| DnaG-F (SEQ ID NO: 8): | |
|---|---|
| ATTAGGTCTCAGCGCCCATCATCATCACCATCATGCTGGACGAATCCCACGC | |
| DnaG-R (SEQ ID NO: 9): | |
| TTAAGGTCTCATATCATTACTTTTTCGCCAGCTCCTGG | |
| dnaG, E.coli primase (SEQ ID NO: 10): | |
| 1 | MASAHHHHHH AGRIPRVFIN DLLARTDIVD LIDARVKLKK QGKNFHACCP FHNEKTPSFT |
| 61 | VNGEKQFYHC FGCGAHGNAI DFLMNYDKLE FVETVEELAA MHNLEVPFEA GSGPSQIERH |
| 121 | QRQTLYQLMD GLNTFYQQSL QQPVATSARQ YLEKRGLSHE VIARFAIGFA PPGWDNVLKR |
| 181 | FGGNPENRQS LIDAGMLVTN DQGRSYDRFR ERVMFPIRDK RGRVIGFGGR VLGNDTPKYL |
| 241 | NSPETDIFHK GRQLYGLYEA QQDNAEPNRL LVVEGYMDVV ALAQYGINYA VASLGTSTTA |
| 301 | DHIQLLFRAT NNVICCYDGD RAGRDAAWRA LETALPYMTD GRQLRFMFLP DGEDPDTLVR |
| 361 | KEGKEAFEAR MEQAMPLSAF LFNSLMPQVD LSTPDGRARL STLALPLISQ VPGETLRIYL |
| 421 | RQELGNKLGI LDDSQLERLM PKAAESGVSR PVPQLKRTTM RILIGLLVQN PELATLVPPL |
| 481 | ENLDENKLPG LGLFRELVNT CLSQPGLTTG QLLEHYRGTN NAATLEKLSM WDDIADKNIA |
| 541 | EQTFTDSLNH MFDSLLELRQ EELIARERTH GLSNEERLEL WTLNQELAKK |

### Example 7

### DNA Amplification with dnaG Primase and Phi29 Polymerase

Whole-genome amplification was performed in 25 μl reactions containing: 50 mM Tris-HCl pH 7.5, 5 mM MgCl₂, 4 mM DTT, 0.6×SYBR green, 0.2 mM dNTP, 3 ng M13 ssDNA, 0.2 mM NTP and 150 μl Phi29. The reaction mixtures contained various amounts of dnaG and the mixtures were incubated at 30° C. overnight in a MiniOpticon (BioRad) with data acquisition every 8 minutes. The fluorescence after overnight incubation, with the fluorescence baseline after first cycle subtracted, was 0.12, 0.22, 0.28, 0.34, 0.26 and 0.25 for reactions with 0, 0.5, 1, 1.5, 2.0 and 2.5 μl dnaG (530 ng/μl), respectively. dnaG stimulated DNA amplification in a dose-dependent manner with maximum amplification at about 800 ng/reaction. These results indicate that dnaG synthesizes primers that can be used by Phi29 polymerase.

### Example 8

### Cloning of Phage Helicase-Deficient T7 gp4 (K318A) Primase

Gene gp4 of the phage T7 encodes a well-characterized protein with both helicase and primase activity (Frick et al. 2001, Annu. Rev. Biochem, 70:39-80). The coding sequence was codon-optimized and the gene was synthesized by Mr Gene, Gmbh (Regensburg, Germany) (SEQ ID NO: 11). Restriction sites for enzyme Eco31I were included in the 5′ and 3′ ends for directional cloning of the gene into the expression vector pKB.

| Nucleotide Sequence of gp4 (SEQ ID NO: 11): | |
|---|---|
| 1 | CATCATCATC ATCACCACGA CAACAGCCAC GATAGCGATT CCGTTTTCCT GTATCACATC |
| 61 | CCGTGTGACA ATTGTGGTTC CTCAGATGGC AATAGCCTGT TCTCAGACGG TCACACCTTT |
| 121 | TGCTATGTGT GTGAGAAATG GACCGCCGGT AATGAGGATA CGAAAGAGCG TGCCTCTAAA |
| 181 | CGTAAACCGA GTGGCGGGAA ACCAATGACC TATAATGTGT GGAACTTCGG CGAAAGCAAT |
| 241 | GGTCGTTATT CTGCCCTGAC TGCCCGTGGG ATTAGTAAAG AAACCTGCCA GAAAGCGGGG |
| 301 | TATTGGATCG CTAAAGTGGA TGGGGTGATG TATCAGGTTG CCGATTATCG TGATCAGAAT |
| 361 | GGGAACATTG TGAGTCAAAA AGTCCGTGAC AAAGACAAAA ACTTCAAAAC AACCGGGAGC |
| 421 | CATAAAAGTG ACGCCCTGTT TGGTAAACAC CTGTGGAATG GGGGTAAGAA AATCGTCGTA |
| 481 | ACCGAGGGTG AAATTGATAT GCTGACAGTA ATGGAGCTGC AGGACTGTAA ATATCCGGTG |
| 541 | GTATCACTGG GACATGGTGC TTCAGCTGCC AAGAAAACAT GTGCCGCCAA CTATGAGTAT |
| 601 | TTCGACCAGT TTGAGCAAAT CATCCTGATG TTCGATATGG ATGAAGCCGG TCGTAAAGCA |
| 661 | GTGGAAGAAG CTGCCCAGGT TCTGCCAGCT GGTAAAGTTC GTGTTGCTGT ACTGCCGTGT |
| 721 | AAAGATGCCA ATGAGTGCCA CCTGAATGGT CATGATCGTG AGATCATGGA ACAGGTCTGG |
| 781 | AACGCTGGTC CTTGGATCCC TGATGGTGTT GTTAGCGCTC TGTCACTGCG TGAGCGTATT |
| 841 | CGTGAGCATC TGTCCAGCGA AGAAAGTGTT GGTCTGCTGT TTAGTGGGTG TACCGGTATT |
| 901 | AATGACAAAA CCCTGGGTGC TCGTGGGGGT GAAGTGATTA TGGTGACCAG TGGTAGCGGT |
| 961 | ATGGGTAAAA GCACGTTTGT TCGCCAGCAA GCACTGCAAT GGGGTACTGC TATGGGCAAG |
| 1021 | AAAGTGGGTC TGGCCATGCT GGAAGAGTCT GTGGAGGAAA CCGCCGAGGA TCTGATTGGA |
| 1081 | CTGCATAACC GTGTACGCCT GCGCCAAAGC GACAGCCTGA AACGTGAAAT CATCGAGAAC |
| 1141 | GGGAAATTTG ATCAGTGGTT CGACGAACTG TTCGGGAATG ACACGTTCCA TCTGTATGAC |
| 1201 | AGCTTTGCCG AGGCAGAAAC CGATCGCCTG CTGGCTAAAC TGGCCTATAT GCGCTCTGGG |
| 1261 | CTGGGTTGTG ACGTGATCAT CCTGGACCAT ATTAGCATTG TGGTGTCCGC TTCAGGAGAG |
| 1321 | TCAGACGAGC GTAAAATGAT TGATAATCTG ATGACCAAAC TGAAAGGCTT CGCCAAATCA |
| 1381 | ACGGGCGTTG TACTGGTGGT AATCTGTCAC CTGAAAAACC CGGACAAAGG CAAAGCACAC |
| 1441 | GAAGAAGGTC GTCCGGTTAG TATCACCGAT CTGCGTGGTA GTGGTGCGCT GCGTCAACTG |
| 1501 | AGCGATACGA TTATTGCTCT GGAGCGTAAC CAGCAAGGGG ATATGCCTAA TCTGGTTCTG |
| 1561 | GTCCGTATTC TGAAATGCCG CTTCACCGGC GATACTGGTA TTGCCGGCTA TATGGAGTAT |
| 1621 | AACAAAGAGA CTGGCTGGCT GGAACCGTCA TCTTATAGCG GCGAGGAGGA GTCTCATTCG |
| 1681 | GAAAGCACGG ATTGGAGCAA CGATACTGAT TTTTGATAAA GCGCTGCACT GAGCTAATGA |
| 1741 | TATGAGACC |

As discussed above, one advantage of using polymerases with strand-displacement activity eliminates the necessity of having a helicase in DNA amplification reactions. This simplifies the WGA reaction in that there is no need to add a dTTP regeneration capability as in reactions by Kong and co-workers (Li et al. 2008, Nucleic Acids Research, 36(13):e79-; US patent application 20050164213).

Without wishing to be bound by any theory, it is thought that the helicase domains of the phage T7 gp4 protein assemble to form a hexameric ring-shaped structure. One of the ssDNA strands is threaded through the hole during helicase-dependent dissociation of the two strands of dsDNA, which is thought to bring six primase domains in close proximity to one another as well as to the ssDNA. Richardson and co-workers have shown that adjacent primase units are important for activity. They postulate that the zinc-binding domain of one primase molecule and the RNA-polymerase domain of an adjacent primase molecule together form an active primase (Lee et al. 2002, Proc. Natl. Acad. Sci. 99(20):12703-12708). The helicase domain essentially acts as a scaffold for bringing primase molecules into close proximity of each other. The T7 helicase utilizes preferentially dTTP as energy source for translocation along DNA. It has been shown that mutating lysine 318 in T7 gp4 to alanine eliminates the dTTPase activity and the helicase activity. However, the primase activity of the K318A mutant is only 1.5-2-fold lower than that of the wild-type (Patel et al. 1994, Biochemistry 33(25): 7857-68).

The K318A mutation was introduced into gp4 by inverse PCR of the vector containing gp4 using phosphorylated primers Heli-K318A-F (SEQ ID NO: 12) and Heli-K318A-R (SEQ ID NO: 13), followed by ligation of the PCR product. The plasmid was digested with Eco31I and the insert was ligated into our expression vector pKB. The amino acid sequence of gp4 K318 is given in SEQ ID NO: 14. An example of expression and purification is given by Patel et al., 1992, J. Biol. Chem. 267(21):15013-15021.

| Heli-K318A-F Primer (SEQ ID NO: 12): | |
|---|---|
| GCGTCGACGTTTGTTCGCCAGCAAGCA | |
| Heli-K318A-R Primer (SEQ ID NO: 13): | |
| ACCCATACCGCTACCACTGGT | |
| Amino Acid Sequence of gp4 K318A (SEQ ID NO: 14): | |
| 1 | MASAHHHHHH DNSHDSDSVF LYHIPCDNCG SSDGNSLFSD GHTFCYVCEK WTAGNEDTKE |
| 61 | RASKRKPSGG KPMTYNVWNF GESNGRYSAL TARGISKETC QKAGYWIAKV DGVMYQVADY |
| 121 | RDQNGNIVSQ KVRDKDKNFK TTGSHKSDAL FGKHLWNGGK KIVVTEGEID MLTVMELQDC |
| 181 | KYPVVSLGHG ASAAKKTCAA NYEYFDQFEQ IILMFDMDEA GRKAVEEAAQ VLPAGKVRVA |
| 241 | VLPCKDANEC HLNGHDREIM EQVWNAGPWI PDGVVSALSL RERIREHLSS EESVGLLFSG |
| 301 | CTGINDKTLG ARGGEVIMVT SGSGMGASTF VRQQALQWGT AMGKKVGLAM LEESVEETAE |
| 361 | DLIGLHNRVR LRQSDSLKRE IIENGKFDQW FDELFGNDTF HLYDSFAEAE TDRLLAKLAY |
| 421 | MRSGLGCDVI ILDHISIVVS ASGESDERKM IDNLMTKLKG FAKSTGVVLV VICHLKNPDK |
| 481 | GKAHEEGRPV SITDLRGSGA LRQLSDTIIA LERNQQGDMP NLVLVRILKC RFTGDTGIAG |
| 541 | YMEYNKETGW LEPSSYSGEE ESHSESTDWS NDTDF** |

### Example 9

### DNA Amplification with gp4 K318A Primase, T7 DNA Polymerase and Phi29 Polymerase

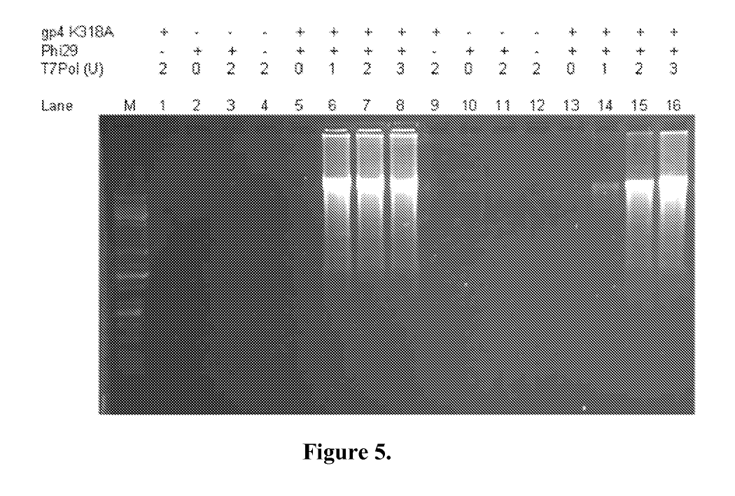

Whole-genome amplification was performed in 25 μl reactions containing: 20 mM Tris-glutamate pH 7.5, 6 mM DTT, 10 mM MgCl₂, 100 mM potassium glutamate, 0.05 mg/ml BSA, 0.6× SYBR green, 1 mM dNTP, 0.2 mM NTP, 1 ng M13 ssDNA and 0.025 U yeast pyrophosphatase (Fermentas). Some reactions further contained 160 ng gp4 K318 and/or 150 ng Phi29 polymerase and/or 0, 1, 2 or 3 U T7 DNA polymerase (Fermentas). Reactions 1-8 were pre-incubated for 20 minutes at 25° C. in the presence of primase before adding polymerases at 4° C. followed by overnight incubation in a RotorGene thermocycler (Corbett Life Science). In reactions 9-16 all enzymes were added at the same time at 4° C. and then incubated at 30° C. overnight. The amplification products were run on a 1% agarose gel (FIG. 5). The result shows a strong amplification of DNA in the presence of Phi29, T7 DNA polymerase and gp4 K318A primase. Leaving out one of the three enzymes resulted in no amplification visible on the gel.

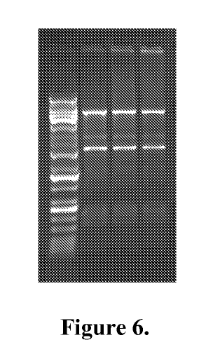

The WGA reactions were tested for specificity of amplification by both restriction digest and by qPCR. The DNA amplification products from reactions 6-8 and 14-16 were heat-inactivated by incubating the samples for 15 minutes at 75° C. The samples were digested with MboI and run on an agarose gel. Bands of the expected sizes for MboI-digested M13 mp18 DNA were observed on the gel (FIG. 6).

The specificity and extent of amplification was further determined using qPCR. qPCR reactions were performed using Kapa SYBR Fast Universal with 0.2 uM of each of primers M13-20 (SEQ ID NO:6) and M13 reverse (SEQ ID NO:7). Two μl of a 1000-fold dilution of WGA reactions were added to each 20 μl qPCR reaction. A standard curve with 10-fold dilutions of M13 DNA from 1 ng to 10 fg was included. The following cycling protocol was used: 2 minutes at 95° C., followed by 40 cycles of (2 seconds at 95° C., 20 seconds at 60° C., data acquisition) followed by a melt curve. Meltcurve analysis showed that all the samples, except the no template controls, had the same melting temperature. This indicated that the qPCR products are specific. Quantitative analysis showed that DNA in WGA reactions with Phi29, gp4 K318A and T7 Pol was amplified about 4000-fold resulting in 4.5 μg, 4.7 μg and 4.3 μg for reactions 6, 7 and 8, respectively.

### Example 10

### Amplification of Genomic DNA Using gp4 K318A Primase, T7 DNA Polymerase and Phi29 Polymerase

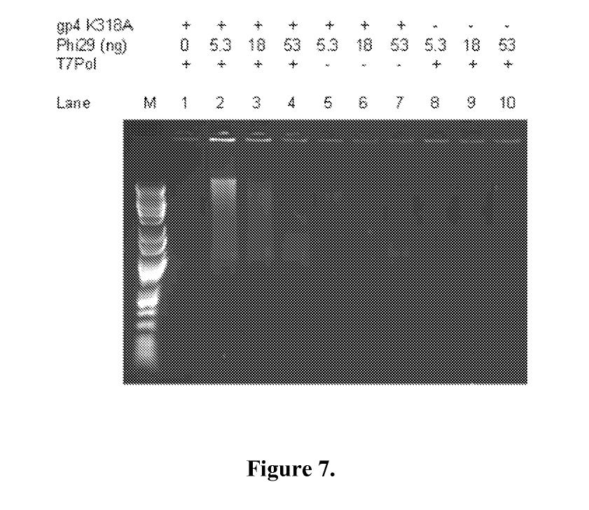

Whole-genome amplification was performed in 25 μl reactions containing: 20 mM Tris-glutamate pH 7.5, 6 mM DTT, 10 mM MgCl₂, 100 mM potassium glutamate, 0.05 mg/ml BSA, 0.6× SYBR green, 1 mM dNTP, 0.2 mM NTP, 30 ng denatured human genomic DNA and 0.025 U yeast pyrophosphatase (Fermentas). The reactions further contained 160 ng gp4 K318A and/or 2 U T7 DNA polymerase, and/or 5.3 ng, 18 ng or 53 ng Phi29 DNA polymerase. The reactions were incubated overnight at 30° C. 6 μl of each reaction was run on an agarose gel. Exemplary amplification results are shown in FIG. 7. The gel shows strong amplification of genomic DNA in the presence of gp4 K318A, Phi29 and T7 DNA polymerases. There is some amplification in reactions with gp4 K318A and Phi29 DNA polymerase.

### Example 11

### DNA Amplification with T7 Primase/Helicase, T7 DNA Polymerase and Phi29 Polymerase

Wild-type gene gp4 of the phage T7 encodes a well-characterized protein with both helicase and primase activity (Frick et al. 2001, Annu. Rev. Biochem, 70:39-80). The DNA amplification reactions are set up by adding 0.5 ug T7-gp4A primase/helicase (Biohelix, Beverly, Mass., USA) per 25 μl reaction volume to a reaction containing 10 ng human genomic DNA or 1 ng M13 DNA, 35 mM Tris-HCl pH 8.0, 50 mM KCl, 10 mM MgCl₂, 5 mM (NH₄)₂SO₄, 1 mM dNTPs, 0.3 mM rATP, 0.4 mM rCTP, 0.5 ug T7 Sequenase, 2 U T7 DNA polymerase (Fermentas), 0.025 U yeast pyrophosphatase (Fermentas, Vilnius, Lithuania), 0.75 ug creatine kinase, 25 ng nucleotide diphosphokinase, 10 mM creatine phosphate and 20 U Phi29 DNA polymerase (Fermentas, Vilnius, Lithuania). The reactions are incubated for 12 hours at 30° C. and run on an agarose gel. Whole genome amplification are observed.

### Example 12

### DNA Amplification with Truncated T7 Primase/Helicase and Phi29 Polymerase

The strong strand-displacement activity of phage Phi29 DNA polymerase eliminates the necessity of having a helicase in isothermal DNA amplification reactions. This simplifies the WGA reaction in that there is no need to add a dTTP regeneration capability as in reactions by Kong and co-workers (US Publication No. 20070254304, US Publication No. 20070207495, US Publication No. 20060154286, and Li et al. 2008, Nucleic Acids Research, 36(13):e79). A C-terminal truncation of T7 gp4, encompassing the N-terminal 271 amino acids but lacking helicase activity encoded by the C-terminal domain, is able to synthesize primers (Frick et al., 1998, Proc. Natl. Acad. Sci. 95:7957-7962).

Two Eco47II restriction sites were included in the codon-optimized full-length gp4 gene that was synthesized, see Example 8. Digestion with Eco47II and re-ligating the large fragment generated a primase construct with the C-terminus deleted. The truncated gp4 (HeliTrunc) was cloned into our expression vector using Eco31I sites flanking the coding sequence. The amino acid sequence of a truncated T7 gp4A primase (HeliTrunc) is given as SEQ ID NO:15. An example of expression and purification is given by Frick et al. 1998, Proc. Natl. Acad. Sci. 95:7957-7962.

| Amino Acid Sequence of Truncated T7 gp4A, HeliTrunc (SEQ ID NO: 15): | |
|---|---|
| 1 | MASAHHHHHH DNSHDSDSVF LYHIPCDNCG SSDGNSLFSD GHTFCYVCEK WTAGNEDTKE |
| 61 | RASKRKPSGG KPMTYNVWNF GESNGRYSAL TARGISKETC QKAGYWIAKV DGVMYQVADY |
| 121 | RDQNGNIVSQ KVRDKDKNFK TTGSHKSDAL FGKHLWNGGK KIVVTEGEID MLTVMELQDC |
| 181 | KYPVVSLGHG ASAAKKTCAA NYEYFDQFEQ IILMFDMDEA GRKAVEEAAQ VLPAGKVRVA |
| 241 | VLPCKDANEC HLNGHDREIM EQVWNAGPWI PDGVVSAALS |

Primase reactions in total volume of 10 μl A were set up containing 20 mM Tris-glutamate pH 7.5, 6 mM DTT, 7 mM MgCl₂, 100 mM potassium glutamate, 0.05 mg/ml BSA, 0.5 mM NTP and 3 ng M13 ssDNA. The reactions further contained various amounts of gp4A HeliTrunc. The reactions were incubated for 10 minutes at 25° C. and then put on ice. 15 μl of the following Phi29 master mix was added to each reaction: 20 mM Tris-glutamate pH 7.5, 6 mM DTT, 2 mM MgCl₂, 100 mM potassium glutamate, 0.05 mg/ml BSA, 1× SYBR Green, 0.3 mM dNTP and 150 ng Phi29. The reactions were incubated overnight at 30° C. in an Eppendorf RealPlex4 Thermocycler. The fluorescence increased faster in reactions with HeliTrunc, compared to reactions without HeliTrunc, suggesting that HeliTrunc stimulates DNA amplification in a dose-dependent manner.

### Equivalents

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. The articles “a”, “an”, and “the” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses variations, combinations, and permutations in which one or more limitations, elements, clauses, descriptive terms, etc., from one or more of the claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth herein. It should also be understood that any embodiment of the invention, e.g., any embodiment found within the prior art, can be explicitly excluded from the claims, regardless of whether the specific exclusion is recited in the specification.

It should also be understood that, unless clearly indicated to the contrary, in any methods claimed herein that include more than one act, the order of the acts of the method is not necessarily limited to the order in which the acts of the method are recited, but the invention includes embodiments in which the order is so limited. Furthermore, where the claims recite a composition, the invention encompasses methods of using the composition and methods of making the composition.

### INCORPORATION OF REFERENCES

All publications and patent documents cited in this application are incorporated by reference in their entirety to the same extent as if the contents of each individual publication or patent document were incorporated herein.

What is claimed is:

1. A method for amplifying nucleic acids, the method comprising a step of:
incubating a template nucleic acid and an amplification mixture comprising a primase and a polymerase having strand-displacement ability such that the template nucleic acid becomes amplified,
wherein the amplification mixture does not contain exogenously-added oligonucleotide primers and does not contain a helicase.

2. The method of claim 1, wherein the amplification mixture does not contain ssDNA binding proteins.

3. The method of claim 1, wherein the amplification mixture does not contain an ATP regeneration system.

4-6. (canceled)

7. The method of any one of the preceding claims, wherein the template nucleic acid comprises genomic DNA.

8-9. (canceled)

10. The method of any one of the preceding claims, wherein the template nucleic acid is obtained from a human biopsy, blood, a forensic sample, and/or a single cell.

11. The method of claim 1, wherein the template nucleic acid is RNA.

12-13. (canceled)

14. The method of claim 1, wherein the template nucleic acid and the amplification mixture are incubated under a thermal cycling condition.

15. The method of claim 1, wherein the primase is selected from the group consisting of ORF904 primase, a primase from Solfolobus solfataricus, p41-p46 primase complex from Pyrococcus furiosus, a primase from Pyrococcus horikoshii, phage T7 primase, phage T4 primase, E. coli dnaG primase, and fragments thereof.

16. The method of claim 1, wherein the polymerase is selected from the group consisting of Phi29 polymerase, Pyrophage 3173 or exonuclease minus version thereof, T7 DNA polymerase or exonuclease minus version thereof, Taq polymerase, Tpol polymerase, KOD polymerase, Vent or DeepVent polymerases, Bst polymerase, KapaHiFi™ DNA polymerase and combination thereof.

17. (canceled)

18. The method of claim 1, wherein the amplification mixture further comprises one or more low-temperature melting reagents.

19. (canceled)

20. The method claim 1, wherein the amplification mixture further comprises a thermoprotectant.

21. (canceled)

22. A composition for amplifying nucleic acid comprising:
a primase;
a polymerase having strand-displacement ability; and
template nucleic acid,
wherein the composition does not contain a helicase or exogenously-added oligonucleotide primers.

23. The composition of claim 22, wherein the composition does not contain ssDNA binding proteins.

24. The composition of claim 22, wherein the composition does not contain an ATP regeneration system.

25. The composition of claim 22, wherein the template nucleic acid comprises genomic DNA.

26. (canceled)

27. The composition of claim 22, wherein the primase is selected from the group consisting of ORF904 primase, a primase from Solfolobus solfataricus, p41-p46 primase complex from Pyrococcus furiosus, a primase from Pyrococcus horikoshii, phage T7 primase, phage T4 primase, E. coli dnaG primase, and fragments thereof.

28. The composition of claim 22, wherein the polymerase is selected from the group consisting of Phi29 polymerase, Pyrophage 3173 or exonuclease minus version thereof, T7 DNA polymerase or exonuclease minus version thereof, Taq polymerase, Tpol polymerase, KOD polymerase, Vent or DeepVent polymerase, Bst polymerase, KapaHiFi™ DNA polymerase and combination thereof.

29. The composition of claim 22, wherein the composition further comprises one or more low-temperature melting reagents.

30. (canceled)

31. The composition of claim 22, wherein the composition further comprises a thermoprotectant.

32. (canceled)
